(19) United States
(12) Patent Application Publication
(10) Pub. No.: US 2017/0313997 A1
(43) Pub. Date: Nov. 2, 2017

(54) Filamentous Fungal Double-Mutant Host Cells

(71) Applicant: Novozymes A/S, Bagsvaerd (DK)

(72) Inventor: Hiroaki Udagawa, Chiba (JP)

(21) Appl. No.: 15/522,535

(22) PCT Filed: Oct. 28, 2015
(86) PCT No.: PCT/EP2015/074989

§ 371 Date: Apr. 27, 2017

(30) Foreign Application Priority Data
Oct. 29, 2014 (EP) 14190952.3

(51) Int. Cl. C12N 9/10; C12P 21/00

(52) U.S. Cl. CPC C12N 9/1051; C12P 21/00; C12Y 204/01183

(57) ABSTRACT

The present invention relates to mutant filamentous fungal host cells producing a polypeptide of interest, wherein two or more polynucleotides encoding two or more polypeptides involved in alpha-glucan synthesis are inactivated; methods of polypeptide production in said mutants as well as methods of constructing such mutants.

### REFERENCE TO A SEQUENCE LISTING

This application contains a Sequence Listing in computer readable form, which is incorporated herein by reference.

### BACKGROUND OF THE INVENTION

A reporter system for the identification of antifungal compounds in filamentous fungi through cell wall stress-induced promoters to drive expression of a reporter protein has been disclosed; one such induced promoter was the Aspergillus niger agsA promoter; the cloning of the full-length agsA gene using degenerate primers as well as the isolation of ags-homologues from other fungi was provided (WO 03/020922). The inactivation of agsA in A. niger has also been described (Damveld R. et al. 2004. Expression of agsA, one of five 1,3-alpha-D-glucan synthase-encoding genes in Aspergillus niger, is induced in response to cell wall stress. Fungal Genetics and Biology 42 (2005) 165-177).

An Aspergillus niger mutant deficient in another of the five 1,3-alpha-D-glucan synthase-encoding genes, agsE, was also disclosed; glucose oxidase, phospholipase A2 and lipase expression was reported to be improved in such a mutant (WO 2014/013074).

### FIELD OF THE INVENTION

The present invention relates to filamentous fungal expression hosts, wherein two or more polypeptides involved in alpha-glucan synthesis are inactivated.

### SUMMARY OF THE INVENTION

The present inventors were interested in increasing the productivity of an enzyme of interest and found—contrary to what had been reported in literature—that AgsA- or AgsE-inactivated single-mutants of Aspergillus niger provided a lower expression of the protease they tested (shown below). However, to their surprise, a double-mutant provided a significantly increased protease expression (shown below).

Accordingly, in a first aspect the invention relates to mutant filamentous fungal host cells producing a polypeptide of interest, wherein two or more polynucleotides encoding two or more polypeptides involved in alpha-glucan synthesis are inactivated, said two or more polypeptides selected from the group consisting of:
(a) a polypeptide involved in alpha-glucan synthesis comprising an amino acid sequence having at least 70% sequence identity with the amino acid sequence of the Aspergillus niger AgsA polypeptide of SEQ ID NO: 3 and a polypeptide involved in alpha-glucan synthesis comprising an amino acid sequence having at least 70% sequence identity with the Aspergillus niger AgsE polypeptide of SEQ ID NO:6;
(b) a polypeptide involved in alpha-glucan synthesis encoded by:
   (i) a genomic polynucleotide comprising a nucleotide sequence having a least 70% sequence identity with the Aspergillus niger agsA genomic nucleotide sequence in SEQ ID NO: 1 and a polypeptide involved in alpha-glucan synthesis encoded by a genomic polynucleotide comprising a nucleotide sequence having a least 70% sequence identity with the Aspergillus niger agsE genomic nucleotide sequence in SEQ ID NO:4, or
   (ii) the cDNA nucleotide sequences of (i) shown in SEQ ID NO: 2 and SEQ ID NO:5, respectively; AND
(c) two polypeptides involved in alpha-glucan synthesis encoded by polynucleotides that hybridize under medium stringency conditions with:
   (I) the Aspergillus niger agsA genomic nucleotide sequence in SEQ ID NO: 1 and the Aspergillus niger agsE genomic nucleotide sequence in SEQ ID NO:4, respectively;
   (II) the cDNA nucleotide sequences of (I) shown in SEQ ID NO:2 and SEQ ID NO:5, respectively, or
   (III) the full-length complements of (I) or (II).

In a second aspect, the invention relates to methods of producing a polypeptide of interest in a mutant filamentous fungal host cell, said method comprising the steps of:
(a) cultivating a mutant filamentous fungal host cell as defined in the first aspect of the invention under conditions conducive for the production of the polypeptide of interest; and, optionally,
(b) recovering the polypeptide of interest.

In a third aspect, the invention relates to methods of constructing a mutated filamentous fungal host cell, said method comprising inactivating two or more polynucleotides in a filamentous fungal host cell encoding two or more polypeptides, respectively, selected from the group consisting of:
(a) a polypeptide involved in alpha-glucan synthesis comprising an amino acid sequence having at least 70% sequence identity with the amino acid sequence of the Aspergillus niger AgsA polypeptide of SEQ ID NO: 3 and a polypeptide involved in alpha-glucan synthesis comprising an amino acid sequence having at least 70% sequence identity with the Aspergillus niger AgsE polypeptide of SEQ ID NO:6;
(b) a polypeptide involved in alpha-glucan synthesis encoded by:
   (i) a genomic polynucleotide comprising a nucleotide sequence having a least 70% sequence identity with the Aspergillus niger agsA genomic nucleotide sequence in SEQ ID NO: 1 and a polypeptide involved in alpha-glucan synthesis encoded by a genomic polynucleotide comprising a nucleotide sequence having a least 70% sequence identity with the Aspergillus niger agsE genomic nucleotide sequence in SEQ ID NO:4, or
   (ii) the cDNA nucleotide sequences of (i) shown in SEQ ID NO: 2 and SEQ ID NO:5, respectively; AND
(c) two polypeptides involved in alpha-glucan synthesis encoded by polynucleotides that hybridize under medium stringency conditions with:
   (I) the Aspergillus niger agsA genomic nucleotide sequence in SEQ ID NO: 1 and the Aspergillus niger agsE genomic nucleotide sequence in SEQ ID NO:4, respectively;
   (II) the cDNA nucleotide sequences of (I) shown in SEQ ID NO:2 and SEQ ID NO:5, respectively, or
   (III) the full-length complements of (I) or (II).

### BRIEF DESCRIPTION OF THE FIGURES

FIG. 1 shows a diagram of the strain construction strategy employed in the examples below.

FIG. 2 shows a schematic map of plasmid pHUda1685.

FIG. 3 shows a schematic map of plasmid pHUda1701.

FIG. 4 shows a schematic map of plasmid pHUda1657.

FIG. 5 shows a schematic map of plasmid pHUda1694.

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Involved in alpha-glucan synthesis: The term “involved in alpha-glucan synthesis” means that a polynucleotide thus involved encodes an enzyme that takes part in the synthesis of alpha-glucan, especially alpha-D-glucan. In the context of this invention, the preferred encoded polypeptides are 1,3 alpha-D-glucan synthases, most preferably AgsA and AgsE from an Aspergillus niger or allelic homologues or variants thereof.

### DETAILED DESCRIPTION OF THE INVENTION

As already outlined in the summary above, the first aspect of the invention relates to mutant filamentous fungal host cells producing a polypeptide of interest, wherein two or more polynucleotides encoding two or more polypeptides involved in alpha-glucan synthesis are inactivated, the second aspect relates to methods of producing a polypeptide of interest in mutant host cells as defined in the first aspect and the third aspect relates to methods of constructing the mutant filamentous fungal host cells.

All aspects of the invention require the inactivation in a filamentous fungal host cell of two or more polynucleotides encoding two or more polypeptides involved in alpha-glucan synthesis, said two or more polypeptides selected from the group consisting of:
(a) a polypeptide involved in alpha-glucan synthesis comprising an amino acid sequence having at least 60% sequence identity with the amino acid sequence of the Aspergillus niger AgsA polypeptide of SEQ ID NO: 3 and a polypeptide involved in alpha-glucan synthesis comprising an amino acid sequence having at least 70% sequence identity with the Aspergillus niger AgsE polypeptide of SEQ ID NO:6; preferably each polypeptide has a sequence identity to SEQ ID NO:3 or 6, respectively, of at least 65%, e.g. at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polypeptides differ by up to 10 amino acids, e.g., 1, 2, 3, 4, 5, 6, 7, 8, 9, or 10, from the polypeptide of SEQ ID NO: 3 or 6, respectively. The amino acid changes may be of a minor nature, that is conservative amino acid substitutions or insertions that do not significantly affect the folding and/or activity of the protein; small deletions, small amino or carboxyl-terminal extensions, such as an amino-terminal methionine residue; a small linker peptide; or a small extension that facilitates purification by changing net charge or another function, such as a poly-histidine tract, an antigenic epitope or a binding domain;
(b) a polypeptide involved in alpha-glucan synthesis encoded by:
   (i) a genomic polynucleotide comprising a nucleotide sequence having a least 60% sequence identity with the Aspergillus niger agsA genomic nucleotide sequence in SEQ ID NO: 1 and a polypeptide involved in alpha-glucan synthesis encoded by a genomic polynucleotide comprising a nucleotide sequence having a least 60% sequence identity with the Aspergillus niger agsE genomic nucleotide sequence in SEQ ID NO:4; preferably each genomic polynucleotide has a sequence identity to SEQ ID NO:1 or 4, respectively, of at least 65%, e.g. at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polynucleotides differ by up to 10 nucleotides, e.g., 1, 2, 3, 4, 5, 6, 7, 8, 9, or 10, from the polynucleotide of SEQ ID NO: 1 or 4, respectively, or
   (ii) the cDNA nucleotide sequences of (i) having a least 60% sequence identity with the Aspergillus niger agsA cDNA nucleotide sequence in SEQ ID NO: 2 and a polypeptide involved in alpha-glucan synthesis encoded by a polynucleotide comprising a nucleotide sequence having a least 60% sequence identity with the Aspergillus niger agsE cDNA nucleotide sequence in SEQ ID NO:5; preferably each cDNA polynucleotide has a sequence identity to SEQ ID NO:2 or 5, respectively, of at least 65%, e.g. at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the polynucleotides differ by up to 10 nucleotides, e.g., 1, 2, 3, 4, 5, 6, 7, 8, 9, or 10, from the polynucleotide of SEQ ID NO: 2 or 5, respectively; AND
(c) two polypeptides involved in alpha-glucan synthesis encoded by polynucleotides that hybridize under very low, low, medium, medium-high, high or very high stringency conditions with:
   (I) the Aspergillus niger agsA genomic nucleotide sequence in SEQ ID NO: 1 and the Aspergillus niger agsE genomic nucleotide sequence in SEQ ID NO:4, respectively;
   (II) the cDNA nucleotide sequences of (I) shown in SEQ ID NO:2 and SEQ ID NO:5, respectively, or
   (III) the full-length complements of (I) or (II). (Sambrook et al., 1989, Molecular Cloning, A Laboratory Manual, 2d edition, Cold Spring Harbor, New York).

In a preferred embodiment of the invention, the two or more polynucleotides encode a polypeptide involved in alpha-glucan synthesis comprising or consisting of an amino acid sequence having at least 70% sequence identity with the amino acid sequence of the Aspergillus niger AgsA polypeptide of SEQ ID NO: 3 or an allelic variant thereof and a polypeptide involved in alpha-glucan synthesis comprising or consisting of an amino acid sequence having at least 70% sequence identity with the Aspergillus niger AgsE polypeptide of SEQ ID NO:6 or an allelic variant thereof. In another embodiment, the polypeptide comprises or consists of the mature polypeptide of SEQ ID NO: 3 or 6.

The polynucleotide of SEQ ID NO: 1 or 2 or a subsequence thereof and the polypeptide of SEQ ID NO: 3 or a fragment thereof, as well as the polynucleotide of SEQ ID NO: 4 or 5 or a subsequence thereof and the polypeptide of SEQ ID NO: 6 or a fragment thereof, may be used to design nucleic acid probes to identify and clone polynucleotides encoding polypeptides involved in alpha-glucan synthesis from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide involved in alpha-glucan synthesis. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes, the carrier material is then used in a Southern blot.

For purposes of the present invention, hybridization indicates that the polynucleotide hybridizes to a labeled nucleic acid probe corresponding to (i) SEQ ID NO: 1 or 4; (ii) the polypeptide coding sequences of SEQ ID NO: 1 or 4; (iii) the cDNA sequences thereof shown in SEQ ID NO:2 or 5; (iv) the full-length complements thereof; or (v) subsequences thereof; under very low to very high stringency conditions. Molecules to which the nucleic acid probe hybridizes under these conditions can be detected using, for example, X-ray film or any other detection means known in the art.

Examples of conservative amino acid substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

### Polynucleotides

The present invention also relates to inactivation of polynucleotides encoding a polypeptide involved in alpha-glucan synthesis, as described herein.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Aspergillus, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the polypeptide coding sequence of SEQ ID NO: 1 or 4; or on the basis of the cDNA thereof in SEQ ID NO: 2 or 5, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Inactivation or Removal of a Polynucleotide Encoding a Polypeptide

The various aspects and embodiments of the present invention all require the inactivation of two or more polynucleotides encoding two or more polypeptides involved in alpha-glucan synthesis in a filamentous fungal host cell.

Such inactivation may be achieved by disrupting or deleting a polynucleotide, or a portion thereof, encoding a polypeptide of the present invention, which results in the mutant cell producing less of the polypeptide than the parent cell when cultivated under the same conditions.

The mutant cell may be constructed by eliminating expression of the polynucleotide using methods well known in the art, for example, insertions, disruptions, replacements, or deletions. In a preferred aspect, the polynucleotide is inactivated. The polynucleotide to be inactivated may be, for example, the coding region or a part thereof essential for activity, or a regulatory element required for expression of the coding region. An example of such a regulatory or control sequence may be a promoter sequence or a functional part thereof, i.e., a part that is sufficient for affecting expression of the polynucleotide. Other control sequences for possible inactivation include, but are not limited to, a leader, polyadenylation sequence, propeptide sequence, signal peptide sequence, transcription terminator, and transcriptional activator.

Inactivation of the polynucleotide may be performed by subjecting the parent cell to mutagenesis and selecting for mutant cells in which expression of the polynucleotide has been eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting no expression of the gene.

Inactivation of the polynucleotide may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame. Such inactivation may be accomplished by site-directed mutagenesis or PCR generated mutagenesis in accordance with methods known in the art. Although, in principle, the inactivation may be performed in vivo, i.e., directly on the cell expressing the polynucleotide to be inactivated, it is preferred that is performed in vitro as exemplified below.

An example of a convenient way to eliminate expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present invention further relates to a mutant cell of a parent cell that comprises a disruption or deletion of a polynucleotide encoding the polypeptide or a control sequence thereof or a silenced gene encoding the polypeptide, which results in the mutant cell producing less of the polypeptide or no polypeptide compared to the parent cell.

The polypeptide-deficient mutant cells are particularly useful as host cells for expression of native and heterologous polypeptides. Therefore, the present invention further relates to methods of producing a native or heterologous polypeptide, comprising (a) cultivating the mutant cell under conditions conducive for production of the polypeptide; and (b) recovering the polypeptide. The term “heterologous polypeptides” means polypeptides that are not native to the host cell, e.g., a variant of a native protein. The host cell may comprise more than one copy of a polynucleotide encoding the native or heterologous polypeptide.

In a preferred embodiment of the invention, the inactivation of the two or more polynucleotides is done by mutation of the two or more polynucleotides and/or their respective promoters; preferably by partial or full deletion of the two or more polynucleotides and/or their respective promoters from the genome of the host cell.

### Nucleic Acid Constructs

The present invention also relates to nucleic acid constructs comprising a polynucleotide encoding a polypeptide of interest according to the present invention operably linked to one or more control sequences that direct the expression of the coding sequence in a suitable host cell under conditions compatible with the control sequences.

The polynucleotide may be manipulated in a variety of ways to provide for expression of the polypeptide of interest. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase Ill, Trichoderma reesei beta-xylosidase, and Trichoderma reesei translation elongation factor.

The control sequence may also be an mRNA stabilizer region downstream of a promoter and upstream of the coding sequence of a gene which increases expression of the gene.

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

In preferred embodiments of the invention, the polypeptide of interest is homologous or heterologous to the host cell; preferably the polypeptide of interest is a secreted polypeptide; more preferably it is a hormone, enzyme, receptor or portion thereof, antibody or portion thereof, or reporter; even more preferably the polypeptide of interest is a hydrolase, isomerase, ligase, lyase, oxidoreductase or transferase; and most preferably the polypeptide of interest is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

### Expression Vectors

The present invention also relates to recombinant expression vectors comprising a polynucleotide encoding a polypeptide of interest as defined herein, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The mutant filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonaturn, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

### Methods of Production

The present invention also relates to methods of producing a polypeptide of the present invention, comprising (a) cultivating a recombinant or mutant filamentous fungal host cell as defined in the first or third aspect of the present invention under conditions conducive for production of the polypeptide; and, optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

The methods used for cultivation and recovery of the product of interest may be performed by methods known in the art.

### EXAMPLES

A. niger Alpha-Glucan Synthase Genes and Gene-Products

The Aspergillus niger alpha-glucan synthase (agsA) gene is shown in SEQ ID NO:1 (genomic DNA) and in SEQ ID NO:2 (cDNA). The amino acid of the encoded AgsA synthase is shown in SEQ ID NO:3.

The Aspergillus niger alpha-glucan synthase (agsE) gene is shown in SEQ ID NO:4 (genomic DNA) and in SEQ ID NO:5 (cDNA). The amino acid of the encoded AgsA synthase is shown in SEQ ID NO:6.

### Materials and Methods

Molecular cloning techniques are described in Sambrook, J., Fritsch, E. F., Maniatis, T. (1989) Molecular cloning: a laboratory manual (2nd edn.) Cold Spring Harbor Laboratory, Cold Spring Harbor, N.Y.

### Enzymes

Enzymes for DNA manipulations (e.g. restriction endonucleases, ligases etc.) are obtainable from New England Biolabs, Inc. and were used according to the manufacturer's instructions.

### Media and Solutions

AMG trace metals solution was composed of 0.3 g of citric acid, 0.68 g of ZnCl₂, 0.25 g of CuSO₄.5H₂O, 0.024 g of NiCl₂.6H₂O, 1.39 g of FeSO₄.7H₂O, 1.356 g of MnSO₄.5H₂O, and deionized water to 1 liter.

COVE-N-gly plates were composed of 218 g of sorbitol, 10 g of glycerol, 2.02 g of KNO₃, 50 ml of COVE salt solution, 25 g of Noble agar, and deionized water to 1 liter.

COVE medium was composed of 342.3 g of sucrose, 20 ml of 50×COVE salts solution, 10 ml of 1 M acetamide, 10 ml of 1.5 M CsCl2, 25 g of Noble agar, and deionized water to 1 liter.

COVE2 medium was composed of 30 g of sucrose, 20 ml of 50×COVE salts solution, 10 ml of 1 M acetamide, 25 g of Noble agar, and deionized water to 1 liter.

COVE-N plates were composed of 342.3 g of sucrose, 20 ml of COVE salt solution, 3 g of NaNO₃, 30 g of Noble agar, and deionized water to 1 liter.

COVE-N top agarose was composed of 342.3 g of sucrose, 20 ml of COVE salt solution, 3 g of NaNO₃, 10 g of low melt agarose, and deionized water to 1 liter.

COVE-N JP plates were composed of 30 g of sucrose, 20 ml of COVE salt solution, 3 g of NaNO₃, 30 g of Noble agar, and deionized water to 1 liter.

COVE salt solution was composed of 26 g of KCl, 26 g of MgSO₄.7H₂O, 76 g of KH₂PO₄, 50 ml of COVE trace metals solution, and deionized water to 1 liter.

COVE trace metals was composed of 0.04 g of Na₂B₄O₇.10H₂O, 0.4 g of CuSO₄.5H₂O, 1.2 g of FeSO₄.7H₂O, 1.0 g of MnSO₄.5H₂O, 0.8 g of Na₂MoO₄.2H₂O, 10 g of ZnSO₄.7H₂O, and deionized water to 1 liter.

LB medium was composed of 10 g of tryptone, 5 g of yeast extract, 5 g of sodium chloride, and deionized water to 1 liter.

LB plus ampicillin plates were composed of 10 g of tryptone, 5 g of yeast extract, 5 g of sodium chloride, 15 g of Bacto agar, ampicillin at 100 μg per ml, and deionized water to 1 liter.

MSS medium was composed of 70 g of sucrose, 100 g of soy bean powder, three drops of pluronic antifoam, and deionized water to 1 liter; pH adjusted to 6.0.

MU-1 glu medium without urea was composed of 260 g of glucose, 3 g of MgSO₄.7H₂O, 6 g of K₂SO₄, 5 g of KH₂PO₄, 0.5 ml of AMG trace metals solution, a few drops of antifoam, and deionized water to 1 liter; pH adjusted to 4.5.

50% Urea was composed of 500 g of urea and deionized water to 1 liter.

YPG medium was composed of 10 g of yeast extract, 20 g of Bacto peptone, 20 g of glucose, and deionized water to 1 liter.

STC was composed of 0.8 M sorbitol, 25 mM or 50 mM Tris pH 8, and 25 mM or 50 mM CaCl₂.

SPTC was composed of 40% polyethyleneglycol 4000 (PEG4000) in STC buffer.

SOC medium was composed of 20 g of tryptone, 5 g of yeast extract, 0.5 g of NaCl, 10 ml of 250 mM KCl, and deionized water to 1 liter.

TAE buffer was composed of 4.84 g of Tris Base, 1.14 ml of Glacial acetic acid, 2 ml of 0.5 M EDTA pH 8.0, and deionized water to 1 liter.

### Purchased Materials

E. coli DH5-alpha (Toyobo) was used for plasmid construction and amplification. The commercial plasmid pBluescript II SK- (Stratagene #212206) was used for cloning of PCR fragments. Amplified plasmid was recovered with Qiagen® Plasmid Kit (Qiagen). Ligation was done with DNA ligation kit (Takara) or T4 DNA ligase (Boehringer Mannheim). Polymerase Chain Reaction (PCR) was carried out with Expand™ PCR system (Boehringer Mannheim). QIAquick® Gel Extraction Kit (Qiagen) was used for the purification of PCR fragments and extraction of DNA fragments from agarose gel.

### Strains

The expression host strain Aspergillus niger C3105 was isolated by Novozymes and it has been genetically modified to disrupt expression of amyloglycosidase activities and alpha-amylase activities followed by introduction of the Aspergillus niger cytosine deaminase gene (fcy1).

### Plasmids

The plasmid pHUda801 was described in example 4 in WO2012160093.

The plasmid pHUda1019 was described in example 2 in WO2012160093.

The plasmid pRika147 for the vector of expression of the enzyme genes was described in example 9 in WO2012160093.

Transformation of Aspergillus niger

Transformation of the parent Aspergillus niger host cell was achieved using the general methods known for transformation in filamentous fungi, as described in Yelton et al., “Transformation of Aspergillus nidulans by using a trpC plasmid,” Proc Natl Acad Sci USA. 1984 March; 81(5):1470-4, as follows:

Aspergillus niger host strain was inoculated to 100 ml of YPG medium supplemented with 10 mM uridine in case the host strain is a pyrG deficient mutant, and incubated for 16 hrs at 32° C. at 80 rpm. Pellets were collected and washed with 0.6 M KCl, and resuspended 20 ml 0.6 M KCl containing a commercial beta-glucanase product (GLUCANEX™, Novozymes A/S, Bagsværd, Denmark) at a final concentration of 20 mg per ml. The suspension was incubated at 32° C. at 80 rpm until protoplasts were formed, and then washed twice with STC buffer. The protoplasts were counted with a hematometer and resuspended and adjusted in an 8:2:0.1 solution of STC:STPC:DMSO to a final concentration of 2.5×10⁷ protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. COVE-N top agarose, the mixture was poured onto the minimum medium and the plates were incubated at 30° C. for 5 days.

### PCR Amplification:

| | |
|---|---|
| 5x PCR buffer (incl.MgCl2) | 20 μl |
| 2.5 mM dNTP mix | 10 μl |
| Forward primer (100 μM) | 1 μl |
| Reverse primer (100 μM) | 1 μl |
| Expand High Fidelity polymerase (Roche) | 1 μl |
| Template DNA (50-100 ng/μl) | 1 μl |
| Distilled water to | 100 μl |

### PCR Conditions:

| | | |
|---|---|---|
| 94° C. | 2 min | 1 cycle |
| 92° C. | 1 min | |
| 55° C. | 1 min | 30 cycles |
| 72° C. | 1-2 min | |
| 72° C. | 7 min | 1 cycle |

### Shake Flask Cultivation for Reporter Enzyme Production

Spores of the selected transformants were inoculated in 100 ml of MSS media and cultivated at 30 C for 3 days. 10% of seed culture was transferred to MU-1 glu medium in lab-scale tanks with feeding the appropriate amounts of glucose and ammonium and cultivated at 34 C for 6 days. The supernatant was obtained by centrifugation. Culture supernatant after centrifugation was used for enzyme assay.

### Southern Hybridization

Mycelia of the selected transformants were harvested from overnight culture in 3 ml YPG medium, rinsed with distilled water. Ground mycelia were subject to genome DNA preparation using FastDNA SPIN Kit for Soil (MP Biomedicals) follows by manufacture's instruction. Non-radioactive probes were synthesized using a PCR DIG probe synthesis kit (Roche Applied Science, Indianapolis Ind.) followed by manufacture's instruction. DIG labeled probes were gel purified using a QIAquick™ Gel Extraction Kit (QIAGEN Inc., Valencia, Calif.) according to the manufacturer's instructions.

Five micrograms of genome DNA was digested with appropriate restriction enzymes completely for 16 hours (40 μl total volume, 4 U enzyme/μl DNA) and run on a 0.8% agarose gel. The DNA was fragmented in the gel by treating with 0.2 M HCl, denatured (0.5M NaOH, 1.5M NaCl) and neutralized (1 M Tris, pH7.5; 1.5M NaCl) for subsequent transfer in 20×SSC to Hybond N+ membrane (Amersham). The DNA was UV cross-linked to the membrane and prehybridized for 1 hour at 42° C. in 20 ml DIG Easy Hyb (Roche Diagnostics Corporation, Mannheim, Germany). The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker.

### Bradford Assay (Whole Protein Determination)

The Bradford assay, a colorimetric protein assay, is based on an absorbance shift of the dye Coomassie Brilliant Blue G-250 in which under acidic conditions the red form of the dye is converted into its bluer form to bind to the protein being assayed. The binding of the dye to the protein stabilizes the blue anionic form. The increase of absorbance at 595 nm is proportional to the amount of bound dye, and thus to the amount (concentration) of protein present in the sample.

Enzyme samples were diluted by distilled water appropriately and were measured their protein amounts by use of Quick Start™ Bradford Protein Assay (Bio-Rad inc.) followed by manufactures' instruction.

### Example 1: Construction of Plasmid pHUda1685, a Vector for Targeted Gene Disruption of Aspergillus niger agsA Gene

Plasmid pHUda1685 was constructed to contain 5′ and 3′ flanking regions for the Aspergillus niger alpha-glucan synthase (agsA) gene separated by the A. nidulans orotidine-5′-phosphate decarboxylase gene (pyrG) as a selectable marker with its terminator repeats, and the human Herpes simplex virus 1 (HSV-1) thymidine kinase gene. The HSV-1 thymidine kinase gene lies 3′ of the 3′ flanking region of the agsA gene, allowing for counter-selection of Aspergillus niger transformants that do not correctly target integration to the agsA gene locus. The plasmid was constructed in several steps as described below.

A PCR product containing the 5′ flanking region of A. niger agsA was generated using the following primers:

Primer agsA1 (sense):
(SEQ ID NO: 7)
5′ ggtggcggccgcctgtgaatagctaccagc
Primer agsA2 (antisense):
(SEQ ID NO: 8)
5′ ctccactagttgctaatgtattatccatga

The desired fragment was amplified by PCR in a reaction composed of approximately 100 ng of genome DNA of Aspergillus niger C3105, 1 μl of Expand High Fidelity polymerase (Roche), 100 μM of primer agsA1, 100 μM of primer agsA2, 5×PCR buffer (incl. MgCl2), 20 μl 2.5 mM dNTP mix (total volume; 100 μl). The reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 2 minutes; 1 cycle at 72° C. for 7 minutes; and a 4° C. hold. The resulting 2,509 bp PCR fragment was purified by 0.8% agarose gel electrophoresis using TAE buffer, excised from the gel, and extracted using a QIAQUICK® Gel Extraction Kit. The purified 2,509 bp PCR fragment was digested by NotI and SpeI.

Plasmid pHUda801 (Example 4 in WO 2012160093 A1) was digested with Not I and SpeI, and purified by 0.8% agarose gel electrophoresis using TAE buffer, where a 9,558 bp fragment was excised from the gel and extracted using a QIAQUICK® Gel Extraction Kit. The 9,558 bp fragment was ligated to the 2,509 bp PCR fragment in a reaction composed of 1 μl of the 9,558 bp fragment, 3 μl of the 2,509 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda801-5′agsA.

A PCR product containing the 3′ flanking region of A. niger agsA was generated using the following primers:

Primer agsA3 (sense):
(SEQ ID NO: 9)
5′ cttctagagtttaaacaaccgcctcagcag
Primer agsA4 (antisense):
(SEQ ID NO: 10)
5′ ttaattaaagaagggcattctatggacgta

The desired fragment was amplified by PCR in a reaction composed of approximately 100 ng of genome DNA of Aspergillus niger C3105, 1 μl of Expand High Fidelity polymerase (Roche), 100 μM of primer agsA3, 100 μM of primer agsA4, 5×PCR buffer (incl. MgCl2), 20 μl 2.5 mM dNTP mix (total volume; 100 μl). The reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 2 minutes; 1 cycle at 72° C. for 7 minutes; and a 4° C. hold. The resulting 2,299 bp PCR fragment was purified by 0.8% agarose gel electrophoresis using TAE buffer, excised from the gel, and extracted using a QIAQUICK® Gel Extraction Kit. The purified 2,299 bp PCR fragment was digested by XbaI and PacI.

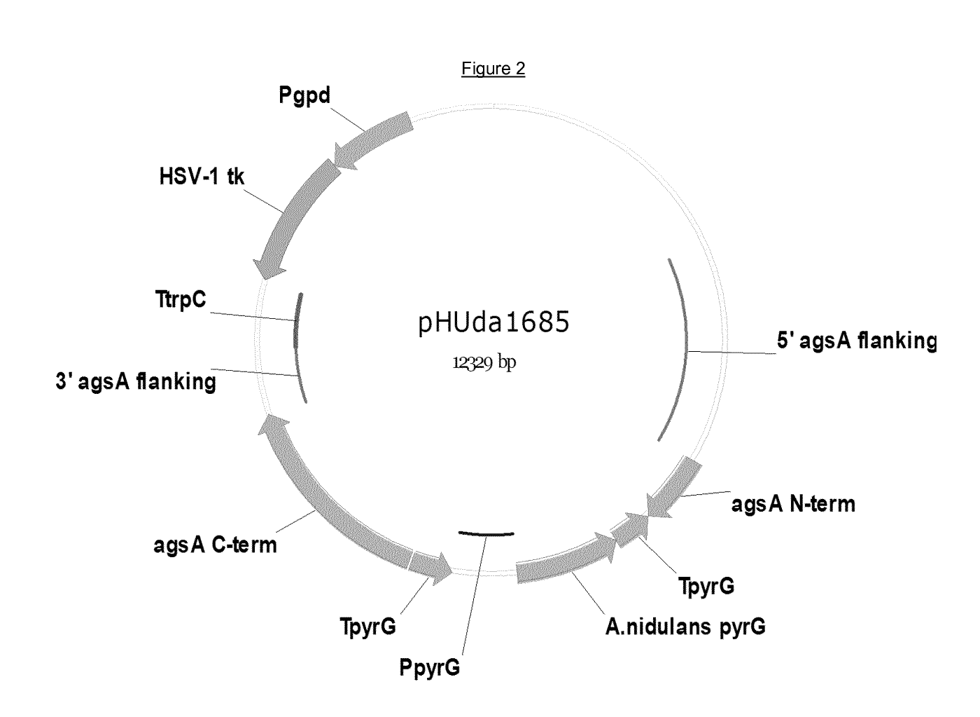

Plasmid pHUda801-5′agsA was digested with XbaI and PacI, and purified by 0.8% agarose gel electrophoresis using TAE buffer, where a 10,030 bp fragment was excised from the gel and extracted using a QIAQUICK® Gel Extraction Kit. The 10,030 bp fragment was ligated to the 2,299 bp PCR fragment in a reaction composed of 1 μl of the 10,030 bp fragment, 3 μl of the 2,299 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda1685 (FIG. 2).

### Example 2: Construction of Plasmid pHUda1701, a Vector for Targeted Gene Disruption of Aspergillus niger agsE Gene

Plasmid pHUda1701 was constructed to contain 5′ and 3′ flanking regions for the Aspergillus niger alpha-glucan synthase (agsE) gene separated by the A. nidulans acetamidase gene (amdS) as a selectable marker, and the human Herpes simplex virus 1 (HSV-1) thymidine kinase gene. The HSV-1 thymidine kinase gene lies 3′ of the 3′ flanking region of the agsE gene, allowing for counter-selection of Aspergillus niger transformants that do not correctly target to the agsE gene locus. The plasmid was constructed in several steps as described below.

A PCR product containing the 5′ flanking region of A. niger agsE was generated using the following primers:

Primer agsE1 (sense):
(SEQ ID NO: 11)
5′ accgcggtctttcagccagggaatggct
Primer agsE2 (antisense):
(SEQ ID NO: 12)
5′ ttactagtacacaatagtgcaggtgatgtc

The desired fragment was amplified by PCR in a reaction composed of approximately 100 ng of genome DNA of Aspergillus niger C3105, 1 μl of Expand High Fidelity polymerase (Roche), 100 μM of primer agsE1, 100 μM of primer agsE2, 5×PCR buffer (incl. MgCl2), 20 μl 2.5 mM dNTP mix (total volume; 100 μl). The reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 2 minutes; 1 cycle at 72° C. for 7 minutes; and a 4° C. hold. The resulting 2,300 bp PCR fragment was purified by 0.8% agarose gel electrophoresis using TAE buffer, excised from the gel, and extracted using a QIAQUICK® Gel Extraction Kit. The purified 2,300 bp PCR fragment was digested by SacII and SpeI.

Plasmid pHUda801 (Example 4 in WO 2012160093 A1) was digested with SacII and SpeI, and purified by 0.8% agarose gel electrophoresis using TAE buffer, where a 9,561 bp fragment was excised from the gel and extracted using a QIAQUICK® Gel Extraction Kit. The 9,561 bp fragment was ligated to the 2,300 bp PCR fragment in a reaction composed of 1 μl of the 9,561 bp fragment, 3 μl of the 2,300 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda801-5′agsE.

A PCR product containing the 3′ flanking region of A. niger agsE was generated using the following primers:

Primer agsE3 (sense):
(SEQ ID NO: 13)
5′ tttctagacgccttaatgatgtggtggtg
Primer agsE4 (antisense):
(SEQ ID NO: 14)
5′ ttaattaaatggcgcccactgctacagg

The desired fragment was amplified by PCR in a reaction composed of approximately 100 ng of genome DNA of Aspergillus niger C3105, 1 μl of Expand High Fidelity polymerase (Roche), 100 μM of primer agsE3, 100 μM of primer agsE4, 5×PCR buffer (incl. MgCl2), 20 μl 2.5 mM dNTP mix (total volume; 100 μl). The reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 2 minutes; 1 cycle at 72° C. for 7 minutes; and a 4° C. hold. The resulting 2,280 bp PCR fragment was purified by 0.8% agarose gel electrophoresis using TAE buffer, excised from the gel, and extracted using a QIAQUICK® Gel Extraction Kit. The purified 2,280 bp PCR fragment was digested by XbaI and PacI.

Plasmid pHUda801-5′agsE was digested with XbaI and PacI, and purified by 0.8% agarose gel electrophoresis using TAE buffer, where a 9,830 bp fragment was excised from the gel and extracted using a QIAQUICK® Gel Extraction Kit. The 9,830 bp fragment was ligated to the 2,280 bp PCR fragment in a reaction composed of 1 μl of the 9,830 bp fragment, 3 μl of the 2,280 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pH Uda801-5′agsE-3′agsE.

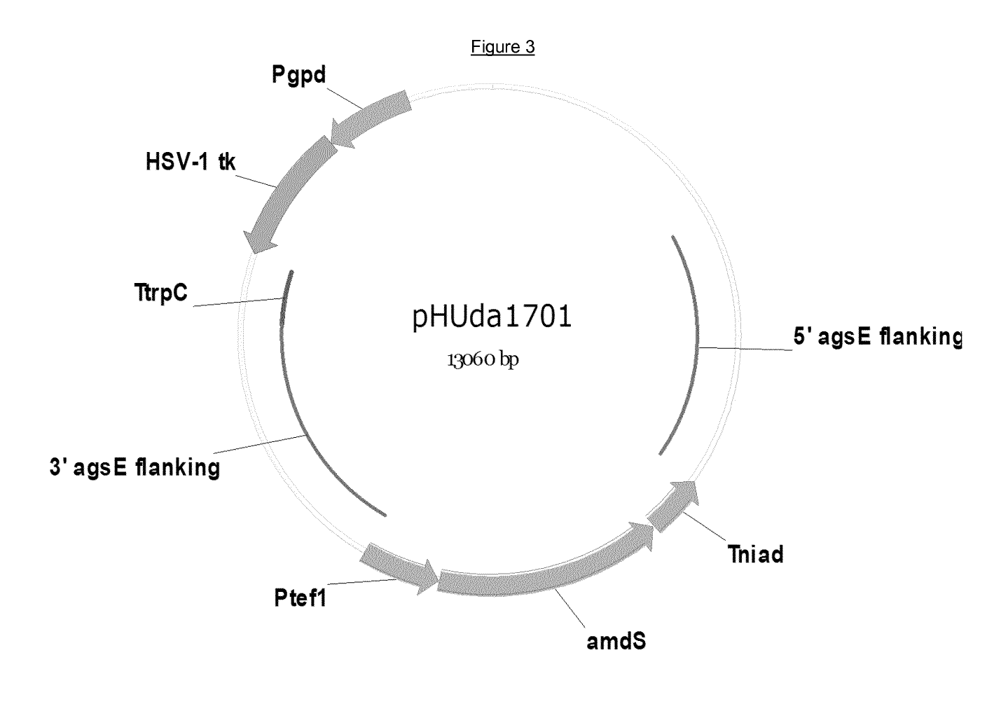

Plasmid pHUda801-5′agsE-3′agsE was digested with XbaI and SpeI, and purified by 0.8% agarose gel electrophoresis using TAE buffer, where a 9,946 bp fragment was excised from the gel and extracted using a QIAQUICK® Gel Extraction Kit. Plasmid pHUda1019 (described in example 2 in WO2012160093) was digested with XbaI and AvrII, and purified by 0.8% agarose gel electrophoresis using TAE buffer, where a 3,114 bp fragment containing amdS gene, A. oryzae tef1 (translation elongation factor 1) promoter and A. oryzae niaD (nitrate reductase) terminator was excised from the gel and extracted using a QIAQUICK® Gel Extraction Kit. The 9,946 bp fragment was ligated to the 3,114 bp fragment in a reaction composed of 1 μl of the 9,946 bp fragment, 3 μl of the 3,114 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda1701 (FIG. 3).

### Example 3: Aspergillus niger C3105 agsA Gene Disruption

The pyrG gene in A. niger C3105 was rescued as follows. The strain C3105 was inoculated on Cove-N JP media containing 10 mM uridine and 1 g/L 5-fluoro-orotic acid (5-FOA) at 30° C. for 5 days. Strains in which the pyrG gene has been deleted will grow in the presence of 5-FOA; those that retain the gene will convert 5-FOA to 5-fluoro-UMP, a toxic intermediate. The grown colonies were transferred with sterile toothpicks to COVE-N-gly plates supplemented with 10 mM uridine and were grown at 30° C. for 7 days. The isolated strain was named M1405.

Protoplasts of Aspergillus niger strain M1405 were prepared by cultivating the strain in 100 ml of YPG medium supplemented with 10 mM uridine at 32° C. for 16 hours with gentle agitation at 80 rpm. Pellets were collected and washed with 0.6 M KCl, and resuspended 20 ml 0.6 M KCl containing a commercial beta-glucanase product (GLUCANEX™, Novozymes A/S, Bagsværd, Denmark) at a final concentration of 20 mg per ml. The suspension was incubated at 32° C. at 80 rpm until protoplasts were formed. Protoplasts were filtered through a funnel lined with MIRACLOTH® into a 50 ml sterile plastic centrifuge tube and were washed with 0.6 M KCl to extract trapped protoplasts. The combined filtrate and supernatant were collected by centrifugation at 2,000 rpm for 15 minutes. The supernatant was discarded and the pellet was washed with 10-25 ml of STC and centrifuged again at 2,000 rpm for 10 minutes and then washed twice with STC buffer. The protoplasts were counted with a hematometer and resuspended and adjusted in an 8:2:0.1 solution of STC:STPC:DMSO to a final concentration of 2.5×10⁷ protoplasts/ml.

Approximately 10 μg of pHUda1685 was added to 0.3 ml of the M1405 protoplast suspension, mixed gently, and incubated on ice for 30 minutes. Three ml of SPTC was added and the protoplast suspension was incubated for 20 minutes at 37° C. After the addition of 12 ml of 50° C. COVE-N top agarose, the mixture was poured onto the COVE-N plates and the plates were incubated at 30° C. for 7 days. The grown transformants were transferred with sterile toothpicks to Cove-N JP plates supplemented with 1.5 μM 5-Fluoro-2-deoxyuridine (FdU), an agent which kills cells expressing the herpes simplex virus (HSV) thymidine kinase gene (TK) harboring in pHUda1685. Single spore isolates were transferred to COVE-N-gly plates.

Possible transformants of Aspergillus niger strain M1405 containing the pHUda1685 to disrupt agsA gene were screened by Southern analysis. Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) follows by manufacture's instruction.

Southern blot analysis was performed to confirm the disruption of the agsA gene locus. Five μg of genomic DNA from each transformant were digested with SpeI. The genomic DNA digestion reactions were composed of 5 μg of genomic DNA, 1 μl of Spe I, 2 μl of 10×NEBuffer 4, and water to 20 μl. Genomic DNA digestions were incubated at 37° C. for approximately 16 hours. The digestions were submitted to 0.8% agarose gel electrophoresis using TAE buffer and blotted onto a hybond N+(GE Healthcare Life Sciences, Manchester, N.H., USA) using a TURBOBLOTTER® for approximately 1 hour following the manufacturer's recommendations. The membrane was hybridized with a 500 bp digoxigenin-labeled Aspergillus niger gasA probe, which was synthesized by incorporation of digoxigenin-11-dUTP by PCR using primers agsA5 (sense) and agsA6 (antisense) shown below.

Forward primer (agsA5):
(SEQ ID NO: 15)
5′ agcagtctcggcggcgagga
Reverse primer (agsA6):
(SEQ ID NO: 16)
5′ attgcccaagcggcgtctca

The amplification reaction (100 μl) was composed of 200 μM PCR DIG Labeling Mix (vial 2) (Roche Applied Science, Palo Alto, Calif., USA), 0.5 μM primers, EXPAND® High Fidelity Enzyme mix (vial 1) (Roche Applied Science, Palo Alto, USA), and 1 μl (100 pg/μl) of pHUda1685 as template in a final volume of 100 μl. The amplification reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 30 seconds and a 4° C. hold. PCR products were separated by 0.8% agarose gel electrophoresis using TAE buffer where a 0.5 kb fragment was excised from the gel and extracted using a QIAQUICK® Gel Extraction Kit. The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker.

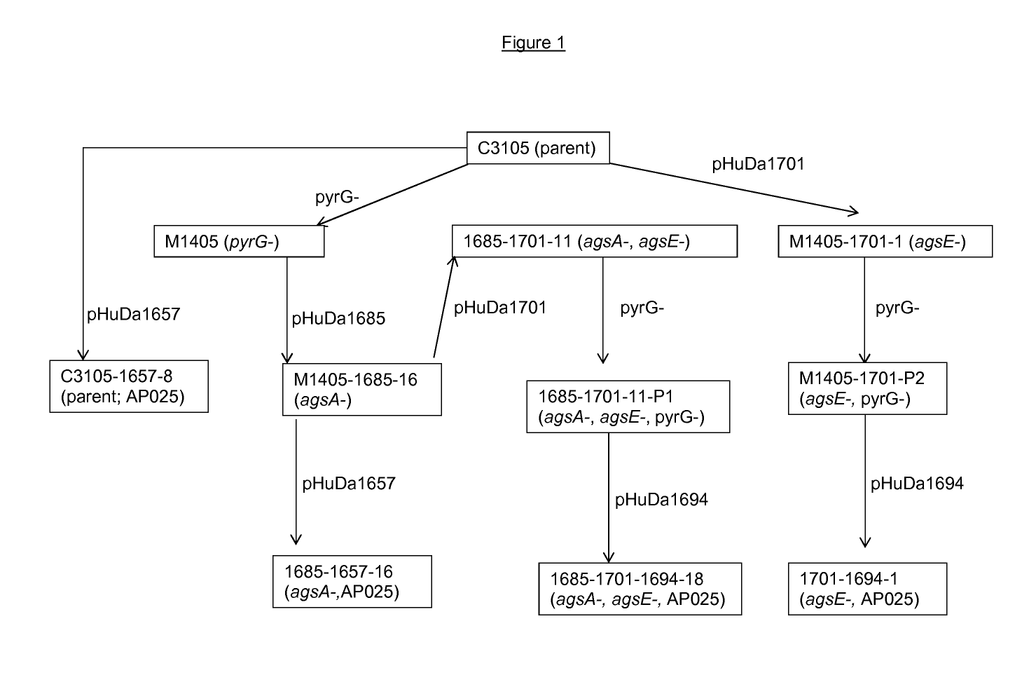

A transformant agsA mutant strain denoted M1405-1685-16 having the correct integration in the agsA locus (a hybridized band shifted from 11.9 kb to 4.7 kb) was selected for the subsequent experiments. See FIG. 1.

### Example 4: The Aspergillus niger agsE Gene Disruption in C3105 and M1405-1685-16

Protoplasts of Aspergillus niger strain C3105 and M1405-1685-16 were prepared by cultivating the strain in 100 ml of YPG medium at 32° C. for 16 hours with gentle agitation at 80 rpm. Pellets were collected and washed with 0.6 M KCl, and resuspended 20 ml 0.6 M KCl containing a commercial beta-glucanase product (GLUCANEX™, Novozymes A/S, Bagsværd, Denmark) at a final concentration of 20 mg per ml. The suspension was incubated at 32° C. at 80 rpm until protoplasts were formed. Protoplasts were filtered through a funnel lined with MIRACLOTH® into a 50 ml sterile plastic centrifuge tube and were washed with 0.6 M KCl to extract trapped protoplasts. The combined filtrate and supernatant were collected by centrifugation at 2,000 rpm for 15 minutes. The supernatant was discarded and the pellet was washed with 10-25 ml of STC and centrifuged again at 2,000 rpm for 10 minutes and then washed twice with STC buffer. The protoplasts were counted with a hematometer and resuspended and adjusted in an 8:2:0.1 solution of STC:STPC:DMSO to a final concentration of 2.5×10⁷ protoplasts/ml.

Approximately 10 μg of pHUda1701 was added to 0.3 ml of the protoplast suspension, mixed gently, and incubated on ice for 30 minutes. Three ml of SPTC was added and the protoplast suspension was incubated for 20 minutes at 37° C. After the addition of 12 ml of 50° C. COVE top agarose, the mixture was poured onto the COVE plates and the plates were incubated at 30° C. for 7 days. The grown transformants were transferred with sterile toothpicks to Cove-2 plates supplemented with 1.5 μM 5-Fluoro-2-deoxyuridine (FdU), an agent which kills cells expressing the herpes simplex virus (HSV) thymidine kinase gene (TK) harboring in pHUda1701. Single spore isolates were transferred to COVE-N-gly plates.

Possible transformants of Aspergillus niger strain C3105 and M1405-1685-16 containing the pHUda1701 to disrupt agsE gene were screened by Southern analysis. Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) follows by manufacture's instruction.

Southern blot analysis was performed to confirm the disruption of the agsE gene locus. Five μg of genomic DNA from each transformant were digested with HindIII and XbaI. The genomic DNA digestion reactions were composed of 5 μg of genomic DNA, 0.5 μl of HindIII, 0.5 μl of XbaI, 2 μl of 10×NEBuffer 4, and water to 20 μl. Genomic DNA digestions were incubated at 37° C. for approximately 16 hours. The digestions were submitted to 0.8% agarose gel electrophoresis using TAE buffer and blotted onto a hybond N+ (GE Healthcare Life Sciences, Manchester, N.H., USA) using a TURBOBLOTTER® for approximately 1 hour following the manufacturer's recommendations. The membrane was hybridized with a 500 bp digoxigenin-labeled Aspergillus niger agsE probe, which was synthesized by incorporation of digoxigenin-11-dUTP by PCR using primers agsE5 (sense) and agsE6 (antisense) shown below.

Forward primer (agsE5):
(SEQ ID NO: 17)
5′ accgggcatgaacatttaac
Reverse primer (agsE6):
(SEQ ID NO: 18)
5′ aagtatttattgtattttgg

The amplification reaction (100 μl) was composed of 200 μM PCR DIG Labeling Mix (vial 2) (Roche Applied Science, Palo Alto, Calif., USA), 0.5 μM primers, EXPAND® High Fidelity Enzyme mix (vial 1) (Roche Applied Science, Palo Alto, USA), and 1 μl (100 pg/μl) of pHUda1685 as template in a final volume of 100 μl. The amplification reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 30 seconds and a 4° C. hold. PCR products were separated by 0.8% agarose gel electrophoresis using TAE buffer where a 0.5 kb fragment was excised from the gel and extracted using a QIAQUICK® Gel Extraction Kit. The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker.

Two strains, M1405-1701-1 and 1685-1701-11, generated from C3105 and M1405-1685-16, respectively, giving the correct integration at the agsE locus (indicated by a hybridized band having shifted from 5.4 kb to 3.2 kb) were selected for the subsequent experiments. See FIG. 1.

### Example 5: Construction of Thermoascus aurantiacus Metal-Protease Gene (ap025) Expression Vector pHUda1657 and pHUda1694

Plasmid pHUda1657 was constructed to contain 2-copies of Thermoascus aurantiacus metal-protease gene (ap025) driven by Aspergillus niger neutral amylase promoter II (Pna2) and glucoamylase terminator (Tamg) put in tandem, the A. nidulans acetamidase gene (amdS) as a selectable marker, and the yeast Saccharomyces cerevisiae FLP recombinase gene (flp) driven by Aspergillus niger acid stable amylase promoter (PasaA) and the Aspergillus oryzae nitrate reductase terminator (Tniad). Plasmid pHUda1694 was an identical construction to pHUda1657 except for having the A. nidulans orotidine-5′-phosphate decarboxylase gene (pyrG) as a selectable marker instead of amdS selective marker. The plasmids were constructed in several steps as described below.

Construction of pHUda1260

The plasmid pHUda1260 was constructed by changing from the A. nidulans orotidine-5′-phosphate decarboxylase gene (pyrG) to the A. nidulans acetamidase gene (amdS) in pRika147.

Plasmid pRika147 (described in example 9 in WO2012160093) was digested with SphI and SpeI, and its ends were filled-in by use of T4 DNA polymerase followed by manufacture's protocol (NEB, New England Biolabs, Inc.). The fragment was purified by 0.8% agarose gel electrophoresis using TAE buffer, where a 9,241 bp fragment was excised from the gel and extracted using a QIAQUICK® Gel Extraction Kit.

Plasmid pHUda1019 (described in example 2 in WO2012160093) was digested with XbaI and AvrII, and its ends were filled-in by use of T4 DNA polymerase followed by manufacture's protocol (NEB, New England Biolabs, Inc.). The fragment was purified by 0.8% agarose gel electrophoresis using TAE buffer, where a 3,114 bp fragment containing amdS gene, A. oryzae tef1 (translation elongation factor 1) promoter and A. oryzae niaD (nitrate reductase) terminator was excised from the gel and extracted using a QIAQUICK® Gel Extraction Kit. The 9,241 bp fragment was ligated to the 3,114 bp fragment in a reaction composed of 1 μl of the 9,241 bp fragment, 3 μl of the 3,114 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda1260. (PWL: Plasmid map and full sequence?)

Construction of Plasmid pHUda1657

A PCR product containing the Thermoascus aurantiacus metal-protease gene (ap025) was generated using the following primers:

Primer ap025-1 (sense):
(SEQ ID NO: 19)
5′ tttggatccaccatgcgtttcatttctgtctcc
Primer ap025-2 (antisense):
(SEQ ID NO: 20)
5′ ccacgtgttagcaaccaaggtatatggcat

The desired fragment was amplified by PCR in a reaction composed of approximately 100 ng of the plasmid DNA harboring ap025 cDNA gene described in WO 2003048353, 1 μl of Expand High Fidelity polymerase (Roche), 100 μM of primer ap025-1, 100 μM of primer ap025-2, 5×PCR buffer (incl. MgCl2), 20 μl 2.5 mM dNTP mix (total volume; 100 μl). The reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 1 minute; 1 cycle at 72° C. for 7 minutes; and a 4° C. hold. The resulting 1,073 bp PCR fragment was purified by 0.8% agarose gel electrophoresis using TAE buffer, excised from the gel, and extracted using a QIAQUICK® Gel Extraction Kit. The purified 1,073 bp PCR fragment was digested by BamHI and PmII.

Plasmid pHUda1260 was digested with BamHI and PmII, and purified by 0.8% agarose gel electrophoresis using TAE buffer, where a 10,512 bp fragment was excised from the gel and extracted using a QIAQUICK® Gel Extraction Kit. The 10,512 bp fragment was ligated to the 1,073 bp PCR fragment in a reaction composed of 1 μl of the 10,512 bp fragment, 3 μl of the 1,073 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda1555.

Plasmid pHUda1555 was digested with SpeI, purified by 0.8% agarose gel electrophoresis using TAE buffer, where an 11,585 bp fragment was excised from the gel and extracted using a QIAQUICK® Gel Extraction Kit.

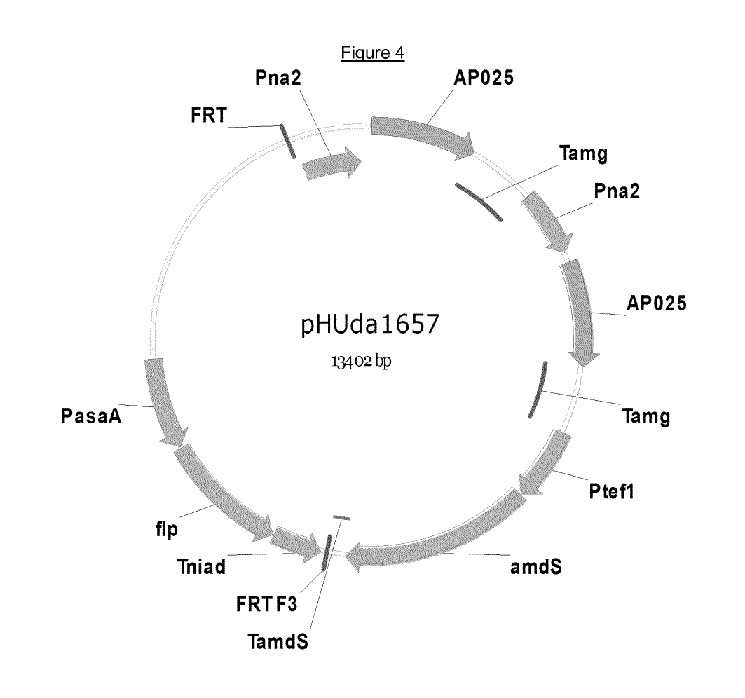

Plasmid pHUda1555 was digested with NheI and SpeI, purified by 0.8% agarose gel electrophoresis using TAE buffer, where a 2,699 bp fragment containing ap025 gene, Aspergillus niger neutral amylase promoter II (Pna2) and glucoamylase terminator (Tamg) was excised from the gel and extracted using a QIAQUICK® Gel Extraction Kit. The 11,585 bp fragment was ligated to the 2,699 bp fragment in a reaction composed of 1 μl of the 11,585 bp fragment, 3 μl of the 2,699 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda1657 (FIG. 4).

Construction of Plasmid pHUda1694

Plasmid pRika147 was digested with NheI and SpeI, purified by 0.8% agarose gel electrophoresis using TAE buffer, where a 7,268 bp fragment was excised from the gel and extracted using a QIAQUICK® Gel Extraction Kit.

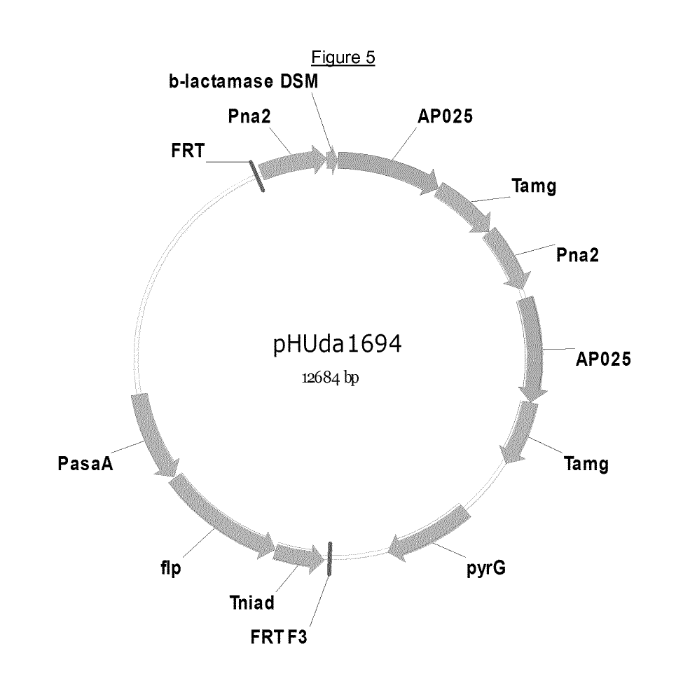

Plasmid pHUda1657 was digested with NheI and SpeI, purified by 0.8% agarose gel electrophoresis using TAE buffer, where a 5,036 bp fragment containing tandem constructs of ap025 gene, Aspergillus niger neutral amylase promoter II (Pna2) and glucoamylase terminator (Tamg) was excised from the gel and extracted using a QIAQUICK® Gel Extraction Kit. The 7,268 bp fragment was ligated to the 5,036 bp fragment in a reaction composed of 1 μl of the 7,268 bp fragment, 3 μl of the 5,036 bp fragment, 1 μl of 5× ligase Buffer, 5 μl of 2× Ligase Buffer and 1 μl of Ligase (Roche Rapid DNA Ligation Kit). The ligation reaction was incubated at room temperature for 10 minutes. Five μl of the ligation mixture were transformed into DH5-alpha chemically competent E. coli cells. Transformants were spread onto LB plus ampicillin plates and incubated at 37° C. overnight. Plasmid DNA was purified from several transformants using a QIA mini-prep kit. The plasmid DNA was screened for proper ligation by use of proper restriction enzymes followed by 0.8% agarose gel electrophoresis using TAE buffer. One plasmid was designated as pHUda1694 (FIG. 5).

### Example 6: Introduction of Thermoascus aurantiacus Metal-Protease Gene (ap025) Expression Vector pHUda1657 in Aspergillus niger Strains C3105 and M1405-1685-16

The ap025 expression plasmids should be introduced at four pre-specified loci which are neutral amylase I (amyA), neutral amylase II (amyB), acid stable amylase (asaA) and putative alkali sulfatase (payA) by flp recombinase.

Protoplasts of Aspergillus niger strain C3105 and M1405-1685-16 were prepared by cultivating the strain in 100 ml of YPG medium at 32° C. for 16 hours with gentle agitation at 80 rpm. Pellets were collected and washed with 0.6 M KCl, and resuspended 20 ml 0.6 M KCl containing a commercial beta-glucanase product (GLUCANEX™, Novozymes A/S, Bagsværd, Denmark) at a final concentration of 20 mg per ml. The suspension was incubated at 32° C. at 80 rpm until protoplasts were formed. Protoplasts were filtered through a funnel lined with MIRACLOTH® into a 50 ml sterile plastic centrifuge tube and were washed with 0.6 M KCl to extract trapped protoplasts. The combined filtrate and supernatant were collected by centrifugation at 2,000 rpm for 15 minutes. The supernatant was discarded and the pellet was washed with 10-25 ml of STC and centrifuged again at 2,000 rpm for 10 minutes and then washed twice with STC buffer. The protoplasts were counted with a hematometer and resuspended and adjusted in an 8:2:0.1 solution of STC:STPC:DMSO to a final concentration of 2.5×10⁷ protoplasts/ml.

Approximately 10 μg of pHUda1657 was added to 0.3 ml of the protoplast suspension, mixed gently and incubated on ice for 30 minutes. Three ml of SPTC was added and the protoplast suspension was incubated for 20 minutes at 37° C. After the addition of 12 ml of 50° C. COVE top agarose supplemented with 50 microg/ml of 5′ fluorocytosine (5FC), an agent which kills cells expressing the Aspergillus niger cytosine deaminase (fcy1) gene harboring in C3105 and M1405-1685-16, the mixture was poured onto the COVE plates and the plates were incubated at 30° C. for 10 days. The grown transformants were transferred with sterile toothpicks to Cove-2 plates supplemented with 10 μg/ml of 5′ fluorocytosine (5FC). Single spore isolates were transferred to COVE-N-gly plates.

Possible transformants of Aspergillus niger strains C3105 and M1405-1685-16 containing the pHUda1657 to introduce ap025 gene were screened by Southern analysis. Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) according to the manufacturers instruction.

Southern blot analysis was performed to confirm the introduction of the ap025 gene at four pre-specified locus (amyA, amyB, asaA, payA). Five μg of genomic DNA from each transformant were digested with HindIII. The genomic DNA digestion reactions were composed of 5 μg of genomic DNA, 0.5 μl of HindIII, 2 μl of 10×NEBuffer 4, and water to 20 μl. Genomic DNA digestions were incubated at 37° C. for approximately 16 hours. The digestions were submitted to 0.5% agarose gel electrophoresis using TAE buffer and blotted onto a hybond N+(GE Healthcare Life Sciences, Manchester, N.H., USA) using a TURBOBLOTTER® for approximately 1 hour following the manufacturer's recommendations. The membrane was hybridized with a 500 bp digoxigenin-labeled ap025 probe, which was synthesized by incorporation of digoxigenin-11-dUTP by PCR using primers ap025-3 (sense) and ap025-4 (antisense) shown below.

Forward primer (ap025-3):
(SEQ ID NO: 21)
5′ aggatcagttcctgctccgg
Reverse primer (ap025-4):
(SEQ ID NO: 22)
5′ ggtatatggcattcgcatag

The amplification reaction (100 μl) was composed of 200 μM PCR DIG Labeling Mix (vial 2) (Roche Applied Science, Palo Alto, Calif., USA), 0.5 μM primers, EXPAND® High Fidelity Enzyme mix (vial 1) (Roche Applied Science, Palo Alto, USA), and 1 μl (100 pg/μl) of pHUda1555 as template in a final volume of 100 μl. The amplification reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 30 seconds and a 4° C. hold. PCR products were separated by 0.8% agarose gel electrophoresis using TAE buffer where a 0.5 kb fragment was excised from the gel and extracted using a QIAQUICK® Gel Extraction Kit. The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker.

Two strains, C3105-1657-8 and 1685-1657-16, generated from parent strains C3105 and M1405-1685-16, respectively, each having the correct integration at the four loci (four hybridized bands at the size of 6.6 kb, 7.0 kb, 8.6 kb and 10.1 kb), were selected for the subsequent experiments. See FIG. 1.

### Example 7: Introduction of Thermoascus aurantiacus Metal-Protease Gene (ap025) Expression Vector pHUda1694 in Aspergillus niger Strains, M1405-1701-1 and 1685-1701-11

The ap025 expression plasmids should be introduced at four pre-specified loci which are neutral amylase I (amyA), neutral amylase II (amyB), acid stable amylase (asaA) and putative alkali sulfatase (payA) by FLP recombinase.

The pyrG gene in M1405-1701-1 and 1685-1701-11 were rescued as follows. Both strains were inoculated on Cove-N JP media containing 10 mM uridine and 1 g/L 5-fluoro-orotic acid (5-FOA) at 30° C. for 5 days. Strains in which the pyrG gene has been deleted will grow in the presence of 5-FOA; those that retain the gene will convert 5-FOA to 5-fluoro-UMP, a toxic intermediate. The grown colonies were transferred with sterile toothpicks to COVE-N-gly plates supplemented with 10 mM uridine and were grown at 30° C. for 7 days. The isolated strains from M1405-1701-1, 1685-1701-11 were named M1405-1701-P2 and 1685-1701-11-P1, respectively.

Protoplasts of Aspergillus niger strains M1405-1701-P2 and 1685-1701-11-P1 were prepared by cultivating the strain in 100 ml of YPG medium supplemented with 10 mM uridine at 32° C. for 16 hours with gentle agitation at 80 rpm. Pellets were collected and washed with 0.6 M KCl, and resuspended 20 ml 0.6 M KCl containing a commercial beta-glucanase product (GLUCANEX™, Novozymes A/S, Bagsværd, Denmark) at a final concentration of 20 mg per ml. The suspension was incubated at 32° C. at 80 rpm until protoplasts were formed. Protoplasts were filtered through a funnel lined with MIRACLOTH® into a 50 ml sterile plastic centrifuge tube and were washed with 0.6 M KCl to extract trapped protoplasts. The combined filtrate and supernatant were collected by centrifugation at 2,000 rpm for 15 minutes. The supernatant was discarded and the pellet was washed with 10-25 ml of STC and centrifuged again at 2,000 rpm for 10 minutes and then washed twice with STC buffer. The protoplasts were counted with a hematometer and resuspended and adjusted in an 8:2:0.1 solution of STC:STPC:DMSO to a final concentration of 2.5×10⁷ protoplasts/ml.

Approximately 10 μg of pHUda1694 was added to 0.3 ml of the protoplast suspension, mixed gently, and incubated on ice for 30 minutes. Three ml of SPTC was added and the protoplast suspension was incubated for 20 minutes at 37° C. After the addition of 12 ml of 50° C. COVE-N top agarose supplemented with 50 μg/ml of 5′ fluorocytosine (5FC), an agent which kills cells expressing the Aspergillus niger cytosine deaminase (fcy1) gene harboring in M1405-1701-P2 and 1685-1701-11-P1, the mixture was poured onto the COVE-N plates and the plates were incubated at 30° C. for 10 days. The grown transformants were transferred with sterile toothpicks to Cove-N JP plates supplemented with 10 microg/ml of 5′ fluorocytosine (5FC). Single spore isolates were transferred to COVE-N-gly plates.

Possible transformants of Aspergillus niger strains M1405-1701-P2 and 1685-1701-11-P1 containing the pHUda1694 to introduce the ap025 gene were screened by Southern analysis. Each of the spore purified transformants were cultivated in 3 ml of YPG medium and incubated at 30° C. for 2 days with shaking at 200 rpm. Biomass was collected using a MIRACLOTH® lined funnel. Ground mycelia were subject to genome DNA preparation using FastDNA SPIN Kit for Soil (MP Biomedicals) follows by manufacture's instruction.

Southern blot analysis was performed to confirm the introduction of the ap025 gene at four pre-specified locus (amyA, amyB, asaA, payA). Five μg of genomic DNA from each transformant were digested with AvrII and HindIII. The genomic DNA digestion reactions were composed of 5 μg of genomic DNA, 0.5 μl of HindIII, 0.5 μl of AvrII, 2 μl of 10×NEBuffer 4, and water to 20 μl. Genomic DNA digestions were incubated at 37° C. for approximately 16 hours. The digestions were submitted to 0.5% agarose gel electrophoresis using TAE buffer and blotted onto a hybond N+(GE Healthcare Life Sciences, Manchester, N.H., USA) using a TURBOBLOTTER® for approximately 1 hour following the manufacturer's recommendations. The membrane was hybridized with a 500 bp digoxigenin-labeled ap025 probe, which was synthesized by incorporation of digoxigenin-11-dUTP by PCR using primers ap025-3 (sense) and ap025-4 (antisense) shown below.

Forward primer (ap025-3):
(SEQ ID NO: 23)
5′ aggatcagttcctgctccgg
Reverse primer (ap025-4):
(SEQ ID NO: 24)
5′ ggtatatggcattcgcatag

The amplification reaction (100 μl) was composed of 200 μM PCR DIG Labeling Mix (vial 2) (Roche Applied Science, Palo Alto, Calif., USA), 0.5 μM primers, EXPAND® High Fidelity Enzyme mix (vial 1) (Roche Applied Science, Palo Alto, USA), and 1 μl (100 pg/μl) of pHUda1555 as template in a final volume of 100 μl. The amplification reaction was incubated in a Bio-Rad® C1000 Touch™ Thermal Cycler programmed for 1 cycle at 94° C. for 2 minutes; 30 cycles each at 94° C. for 30 seconds, 55° C. for 30 seconds, and 72° C. for 30 seconds and a 4° C. hold. PCR products were separated by 0.8% agarose gel electrophoresis using TAE buffer where a 0.5 kb fragment was excised from the gel and extracted using a QIAQUICK® Gel Extraction Kit. The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done followed by manufacture's protocol. The DIG-labeled DNA Molecular Weight Marker II (Roche) was used for the standard marker.

Two strains, 1701-1694-1 and 1685-1701-1694-18, generated from M1405-1701 and 1685-1701-11, respectively, each having the correct integration at the four loci (four hybridized bands at the size of 6.0 kb, 6.3 kb, 7.3 kb and 9.4 kb) were selected for the subsequent experiments. See FIG. 1.

### Example 8: AP025 Protease Expression Evaluation in Shake Flask Culture

The Aspergillus niger strains C3105-1657-8, 1685-1657-16, 1701-1694-1 and 1685-1701-1694-18 were cultivated on COVE-N-gly plates at 30° C. for about a week. A sterile transfer pipette was used to punch a piece of small plugs from each plate, which were each inoculated into 100 ml of MSS medium in 500 ml flasks. The flasks were incubated at 30° C. for 3 days at 200 rpm. And then, 10 ml of culture broth was transferred to 100 ml of MU1 glu medium in 500 ml flasks. The flasks were incubated at 30° C. for 6 days at 200 rpm. Each culture was centrifuged at 5,000 rpm for 10 minutes in a 10 ml test tubes and culture supernatant was recovered for determining protease productivities. The protease productivity assay was performed using a Quick Start™ Bradford Protein Assay Kit (Bio-Rad inc.). Culture supernatants were diluted appropriately in distilled water. A bovine serum albumin (WAKO cat number 519-83921) was diluted using several steps starting with a 0.5 mg/ml concentration and ending with a 0.1 mg/ml concentration in the distilled water. Five μl of each dilution including standard were transferred to a 96-well flat bottom plate. 250 μl of 1× Dye Reagent solution were added to each well and then incubated at room temperature for 5 minutes. The endpoint of the reaction was measured at 595 nm. Whole protein productivities were determined by extrapolation from the generated standard curve and compared to the Aspergillus niger strain C3105-1685-8 set at 100%.

The agsA and agsE double gene disrupted strain gave 23% higher AP025 productivity than a reference strain Aspergillus niger strain C3105-1657-8, even though single agsA or agsE gene disrupted strains showed comparable or a little bit lower protease AP025 productivity than the parent reference strain Aspergillus niger strain C3105-1657-8 in shake flasks (Table 1).

TABLE 1
AP025 protease productivity in shake flask culture.

| | | Relative protein |
|---|---|---|
| Sample Name | Genotype | productivity (%) |
| A. niger C3105-1657-8 | Parent, AP025 | 100% |
| A. niger 1685-1657-16 | agsA-; AP025 | 92% |
| A. niger 1701-1694-1 | agsE-, AP025 | 92% |
| A. niger 1685-1701-1694-18 | agsA-, agsE-, AP025 | 123% |

1. A mutant filamentous fungal host cell producing a polypeptide of interest, wherein two or more polynucleotides encoding two or more polypeptides involved in alpha-glucan synthesis are inactivated, and wherein said two or more polypeptides are selected from the group consisting of:
(a) a polypeptide involved in alpha-glucan synthesis comprising an amino acid sequence having at least 70% sequence identity with the amino acid sequence of the Aspergillus niger AgsA polypeptide of SEQ ID NO: 3 and a polypeptide involved in alpha-glucan synthesis comprising an amino acid sequence having at least 70% sequence identity with the Aspergillus niger AgsE polypeptide of SEQ ID NO:6;
(b) a polypeptide involved in alpha-glucan synthesis encoded by:
(i) a genomic polynucleotide comprising a nucleotide sequence having a least 70% sequence identity with the Aspergillus niger agsA genomic nucleotide sequence in SEQ ID NO: 1 and a polypeptide involved in alpha-glucan synthesis encoded by a genomic polynucleotide comprising a nucleotide sequence having a least 70% sequence identity with the Aspergillus niger agsE genomic nucleotide sequence in SEQ ID NO:4, or
(ii) the cDNA nucleotide sequences of (i) shown in SEQ ID NO: 2 and SEQ ID NO:5, respectively; an
(c) two polypeptides involved in alpha-glucan synthesis encoded by polynucleotides that hybridize under medium stringency conditions with:
(I) the Aspergillus niger agsA genomic nucleotide sequence in SEQ ID NO: 1 and the Aspergillus niger agsE genomic nucleotide sequence in SEQ ID NO:4, respectively;
(II) the cDNA nucleotide sequences of (I) shown in SEQ ID NO:2 and SEQ ID NO:5, respectively, or
(III) the full-length complements of (I) or (II).

2. The mutant filamentous fungal host cell of claim 1, which is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

3. The mutant filamentous fungal host cell of claim 1 which is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

4. The mutant filamentous fungal host cell of claim 1, wherein the polypeptide of interest is homologous or heterologous to the host cell.

5. The mutant filamentous fungal host cell of claim 1, wherein the inactivation of the two or more polynucleotides is done by mutation of the two or more polynucleotides and/or their respective promoters.

6. The mutant filamentous fungal host cell of claim 1, wherein the two or more polynucleotides encode a polypeptide involved in alpha-glucan synthesis comprising or consisting of an amino acid sequence having at least 70% sequence identity with the amino acid sequence of the Aspergillus niger AgsA polypeptide of SEQ ID NO: 3 and a polypeptide involved in alpha-glucan synthesis comprising or consisting of an amino acid sequence having at least 70% sequence identity with the Aspergillus niger AgsE polypeptide of SEQ ID NO:6.

7. A method of producing a polypeptide of interest in a mutant filamentous fungal host cell, said method comprising the steps of:
(a) cultivating the mutant filamentous fungal host cell of claim 1 under conditions conducive for the production of the polypeptide of interest; and, optionally,
(b) recovering the polypeptide of interest.

8. A method of constructing a mutated filamentous fungal host cell, said method comprising inactivating two or more polynucleotides in a filamentous fungal host cell encoding two or more polypeptides, respectively, selected from the group consisting of:
(a) a polypeptide involved in alpha-glucan synthesis comprising an amino acid sequence having at least 70% sequence identity with the amino acid sequence of the Aspergillus niger AgsA polypeptide of SEQ ID NO: 3 and a polypeptide involved in alpha-glucan synthesis comprising an amino acid sequence having at least 70% sequence identity with the Aspergillus niger AgsE polypeptide of SEQ ID NO:6;
(b) a polypeptide involved in alpha-glucan synthesis encoded by:
(i) a genomic polynucleotide comprising a nucleotide sequence having a least 70% sequence identity with the Aspergillus niger agsA genomic nucleotide sequence in SEQ ID NO: 1 and a polypeptide involved in alpha-glucan synthesis encoded by a genomic polynucleotide comprising a nucleotide sequence having a least 70% sequence identity with the Aspergillus niger agsE genomic nucleotide sequence in SEQ ID NO:4, or
(ii) the cDNA nucleotide sequences of (i) shown in SEQ ID NO: 2 and SEQ ID NO:5, respectively; and
(c) two polypeptides involved in alpha-glucan synthesis encoded by polynucleotides that hybridize under medium stringency conditions with:
(I) the Aspergillus niger agsA genomic nucleotide sequence in SEQ ID NO: 1 and the Aspergillus niger agsE genomic nucleotide sequence in SEQ ID NO:4, respectively;
(II) the cDNA nucleotide sequences of (I) shown in SEQ ID NO:2 and SEQ ID NO:5, respectively, or
(III) the full-length complements of (I) or (II).

9. The method of claim 8, wherein the filamentous fungal host cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

10. The method of claim 8, wherein the filamentous fungal host cell produces a polypeptide of interest.

11. The method of claim 8, wherein the inactivation of the two or more polynucleotides is done by mutation of the two or more polynucleotides and/or their respective promoters.

12. The mutant filamentous fungal host cell of claim 4, wherein the polypeptide of interest is a secreted polypeptide.

13. The mutant filamentous fungal host cell of claim 4, wherein the polypeptide of interest is a hormone, enzyme, receptor or portion thereof, antibody or portion thereof, or reporter.

14. The mutant filamentous fungal host cell of claim 13, wherein the enzyme is a hydrolase, isomerase, ligase, lyase, oxidoreductase or transferase.

15. The mutant filamentous fungal host cell of claim 5, wherein the inactivation is by partial or full deletion of the two or more polynucleotides and/or their respective promoters from the genome of the host cell.

16. The method of claim 10, wherein the polypeptide of interest is a secreted polypeptide.

17. The method of claim 10, wherein the polypeptide of interest is a hormone, enzyme, receptor or portion thereof, antibody or portion thereof, or reporter.

18. The method of claim 17, wherein the enzyme is a hydrolase, isomerase, ligase, lyase, oxidoreductase or transferase.

19. The method of claim 11, wherein the inactivation is by partial or full deletion of the two or more polynucleotides and/or their respective promoters from the genome of the host cell.


SEQUENCE LISTING (24 sequences)

SEQ ID NO: 1 - DNA, 7368 bp, aspergillus niger
atgttcggcacgacggttcagcgctgcgtggtgctcattctggggatactcagcgtcacgaccgtgggctggccctacgatgagtcgctcgttgattacaacctgaacgagaacaagaccgcagagggaccgataaactattggggagaatggccagatcacacctatcatccgtcaccagacaactggcgcttccccatctataccatcttcctggatcgcatcgccaacggagatcccgctaatgacgatattaacggcaccacctacgagcatgtactcgattcgaatcagatgcgccatggcggtgacctagtcggcttgattgacacgctcgattacatcagaggcatgggcttcaaggtgagtctattccattgatatcagttcccggtccatccttgactgactgactgatcattctagggaatctacttcgctggtacgggcttgatgaatcttccctggggctacgatggttactccccggtggataccaccctcctcgacaagcaccatggcaccctgtccgactggaggcggaccatcaaggagatccacgaccgggatatgtatgtgatcatggataatacattagcaacgtgagtatgcaccgagtattctagacgaccatgccactaacaatctccccgtgcagtttgagtaacctgatcggtttcaagggccacttgaacgattcggctgatttcaatgcgaaagagtacgaggtggagtacgtcaccgagagaacgtacgccgatttccagttcagcaatgactacaatgacacctgcaactacccgaaattctggaacgagacgggatacccgttgacgtccggtggcgtgcaagacctgaagggttgctacaacagtgatttcgatcagtatggcgaactcgaggcgtttggtaatttccccgactggaagcgacagctcacgaaattcgcttcggtgcaagatcgtctgcgagaatggcacaagccggttcgcgatgtcatcacccggcattcctgcattgtgatcgccagtttggacattgatggcttccggtttgataaggccgtccaggccactctggatccgctgggtgacatgctagcggtctaccgtgaatgtgccaagcaatatggcaagaagaacttcttcctgccgggagagatcacttcgggaaacacctttggtagtctctatcttggccggggtcgtcagccaaaccagacgcccgattcggcagacgctggcgcgaagttgacgaatgcttcgtcggattcctacttcctccgcgatgatggcctgcaggcgttggacgctgcagcctttcactacaccatctatcgttcgatgacccgtttcctgggaatggacgggaacctggtggccggctttgacctgcccactgatttcatcgaagcctggaatggcatgctcgtcactaacgacttcatcaatgccttcacgggcgagttggatcccagacatatgtatggagtgtcgaaccaggataactttcgctggcccgccatcaagaatggtacggagaaatacctgctgggtctatacattgttacgctggagctgccgggaatccctttggttctctggggcgaggagcaggagatgtacgtgttcgatgcgactgcgtccaactatctgttcggtcggcaacccatgacttaccagacggcgtggtggacgcatggctgcttctcgctaaacacctcgaaattctacgatttccccaacgacaagggattgaatggttgcaacgatatcacagtgacttacgaccaacggaatccggcgcacccccttcgcaacatcatgaagcgcatgtttgagattcgggagcagtacccagtggccaatgatggcttcaaccttgagaccatttcccagcttacccaggatatctatcttccgggttcatcgactaccccgacggtgactggtctctggtcagtgctgcgcagctactaccccggggttcagaaagaggcctcgagtagcaacgatactctttggcttgtatatcataatgccaataaaacggaaacctatggcaagaactgtactagcaaggactccgctcttctgtccccttacgagtcgggcacgaagttgaaaaacctcttctatccctacgatgaactcacgctgcaggatggaccaagtgacgctagcaacggcaccgagtcctacgggtgcacgaccaacatgaaattgctgccatgggaattccgggcttacgtcaaggcgagcgacttcgttgaacctggccctaccgtcaccgagtttgtccccggccacgatgctcgcctgctgtcttccgaggatactggggaaacactgaagatccagctgggctactccaaggccatggactgcagtgccatcacgaaagcagtctccctgaattcgaccaccgtcaagggcgtcaatgccacccttgacacctctagcgtgagctgcaccaacgtcaccgccaggacgagcagtaataactacatcggcgaggttcccactgtttggacctggtccgcgaacctgacaaacgtctatcacggaatccaccagctcaccgtcaagaatgcctccacgacgtctggaacgcgcgtagatgccatcgaccgctttttattccgagtcggaacccacaccaacccgttaatatcgccactggccaactactctaccagcctcgtccatcagtcggataacggcagctactatgttcagcatgatgcggccggcgctgaccagttccgctacacgaccgactttggcctgaactggtccaactggacagactacactggtggtaacacgacgattgaatttccgacttggaccggcacagatgctcaaaagtgggagggcactcacattcgcgtccagtatttctcccgccttaccggcagcagtgattacatccaggagggtgattatgactgggaatccggtgtccctcgcagatttccccatctgtggtggaatggcccgtacaaccaatacggttatgatgccggtctggacagcaagatgcggtttgatactaaggatcgccgctggaaatatgatttcgtttatgaatggccgtccgtcggacagatcagtgtctggggcactggcaaggacggtgttccggagacaacgaatgtttatggcgatgttgacaactcatcggttgtacaggtcttgcctccctcctacttgtcgtcgaacgtgattaatatcaccgagctgccccccttcccgcaccttggctggaccatctctctcaatgatgccaacctccgctatgaattgcttccggtcggatctggatgggcccagctggtactttacatccttctctgggtcgtccctatcttgatggggctggcgggagttttcatcttcatccgcaccttctaccgtgttgagctgaataccgatggcaatgttgttaaggaggacaagctgccgctgttgttctggcgcaggatgaagggccaatctggaggtgatggccagatggaccgggaagaatcagatgtcgccgccgtggcaggcgaaatggccattgcaggagcccccgagaaacgccgcacggtcttgatcgccaccatggagtacgatattgcggattggcaggccaaggtcaagattggtggtctgggagtcatggcccagctcatgtcccaaaacctgggatatcagaatcttatctgggtggttccctgcgtgggagacatcgaatatcccgaggataccccgacagagccatacatcgtgaagatcctcgacaacccctatctcgtcaatgtgcagtatcacgttctcaacaatatcacctacgtccttcttgacgctcctgtgttccgccagcagacaaaagccgagccgtatcctccccgcatggatgacctggatagcgccatttactactccgcctggaaccaatgtatcgcagagaccatcaagcgcttcccgtccatcgatctgtatcacatcaacgatttccacggatgcctggcaccgttgtacctcctgcccacacgcacgatccccgtgtgtttgtctctccacaacgccgagttccagggtctctggcccctccggacccagcaagaaaagaaggaagtctgttcggtcttcaacctccccgtcgagacggctaccaaatattgtcaattcggtaatgtgttcaacttgcttcacacaggagcgagttaccttcggttccatcagcgcgggtttggcgcggtcggtgtgtcaaagaagtacggaaagcgttcctgggctcggtatcctattttctggagtttggacaagatcggtagtctgcccaaccctgacccaactgatacggcggccctggaggatactcctgacgagaaagctctgactcggtcatatgaggaacggatcactgacaagctcgaggcccagaagtgggctggcctgaccgaggatcgcaacgccgacctcctggtgttcgtcggtcgctggtctaagcagaagggtgtcgatctgattgcggatgtcatccccgccgttctgtctgaccggccgcaggtgcaggtgatctgtgttggtcccatcatcgatctttacggaagactggctgccattaagctggagaagatcgctgccatgttccctggccgggtcttctcccgacctgagttcactgctttgcccccgtgtgtcttctctggcgctgacttcgctctgatcccgtctcgtgatgagccatttggattgattgccgttgagttcggtcggaagggtgcactgggaatcggatctcgcatcggaggtttgggtcagatgccaggctggtggtacaccgttgaatcagacgccactcgccatcttctacaccagctgaagactgcgatcaaatctgccctggattcgactcctgaaactcgagcccagatgcgcgcaaactccgctaagcagcgattccccgttctcgagtggatccagaaactcgagactttgcagcggacatcgatcaagattcatcatgacaagaacaaggacaccgtgactggttcgatcccggagtctgagagcgtatgggacctgcaaaaccttcagagtgcgcgctattccagtgtggcacttcctaatatggctctgtccagagcggacggcacggaaaccccgcctgaagccatgatccagcaggcccacgctcgccttcaagagatccaggcatccgagggaagcagtcgcgacagcagcctcaaccgcaaactgtcactgggtcgccggtccggccctggtcagggcagaaagcgcttgacgaagaggcaagcgaccgcaagccaggcatccactgatcacggagaagatgagaacgataccgacgctgacgacgaccacaccaggccgcaggaggattacatttctcccgaggaggccatgcaggccgttaacaacactctaactccccatgctatcactggagacagtactccccgcgcctcctacttgtctagacccgtatccccgtaccccatctcacagatctctactccagcgtgcccgacaccgccacccatgactcacttccggaccgtttctatgctgtcgttgccatccgttgttggggaccataaccgcctcagcagtctcggcggcgaggagcagaaccagcccgtcttcgagttgcagaaggtggacccgactttcacggacagcttgggccacttcacccggcgcttcgagcagtcgcttgagaagttgaacaagaagaactccatcacggactattgcattgaggtctatctgatgaagagtgagcgcaagttctacaattcatacaacgacgcccagcttaagaagcagcctaagaaccgttcgctcgcggttcccagctctgagagtgttgtgcaagaagggcccggtggctcagtcgttcgtcactcgtactccagtgaggattctgaaatcaatggggaggatgagattgaccgctggctgatgcggttgggttacacgcgtccgatcgcagttcagcgattcatgagacgccgcttgggcaattggccagtgtattcactattcctgggtctgggtcagatcatcgccaccaactccgcacaaatcaccctgcttgtcggccaggtcggagagaccgctgtgaagctttatgtcatcgccgctatctactgcctgtcctccatcgcgtggtggttcctgtatcatcggttcccagccgtcatctccctcagtcttccctggttcatctactgcatggcattcatcgttatcggtgtttcacccttcggtctcacggctctcggtcgggcctgggctcagaatgttgcggctggcgtctacgccattgcgtcgtctagcggttccttgttcttcgctctgaactttggagaccagggtgccgtccctgtcaaggactggatgttccgcgccagtatcattcaaggtatctcacagatctacacggtcgcgctctggttctggagctccaaggtcactgccgccgaagtgggaggtgtatctgtagcggccttgagctcctggcgtctcacggccgtggtgatgcccattgctgccctgtgcttcgtgatcggagtcctgctggccctgggattgcccaactactaccgtcaagctcccagccgtatcctgttcttctacaagtctctgttccgccgacgcatcatcctgtggttcttcttcatggtcatcgtccagaactggttccttgctgccgctttcggccgcaactggtccttcctctggtcgtccaaacacaccaagccctgggagattgtcctccttgtgttcggcttcttcgtcgtcctatgggtagtcattcttgtcatattccgtgccctgtcgaaggaacacagctggatcttgccagttttcggtctgagtctgggtgctccccggtgggcacaaacctggtggggaacctccaacatcggctactatctcccttgggctggcagcttggtgtcggggtcgatcgcttcccgctgcctgtggctctggctgggactccttgacgagatccaacaagtcggattgggtatgattctgctgcagaccatgactcgcgtgcatgtctgcttcgtcctgttggccgcgcaagcactgggatccatcgccaccatctgcgcccgcggcttcgcgccgaacaagattggccctggggatatctctccggatgtgggcacgtcggtggacaaggtcggtaacgcatggttctggatcgctctcttcttccagctcctggcaaggtaagccttcccacactttcttttctcacgcgagctttatctaacgtttgaacagttggggcttcctgctcttctaccgccgcgaacagctcaaccgaccctaa

SEQ ID NO: 2 - DNA, 7194 bp, aspergillus niger
  CDS (1)..(7191): AgsA encoding cDNA
atgttcggcacgacggttcagcgctgcgtggtgctcattctggggatactcagcgtcacgaccgtgggctggccctacgatgagtcgctcgttgattacaacctgaacgagaacaagaccgcagagggaccgataaactattggggagaatggccagatcacacctatcatccgtcaccagacaactggcgcttccccatctataccatcttcctggatcgcatcgccaacggagatcccgctaatgacgatattaacggcaccacctacgagcatgtactcgattcgaatcagatgcgccatggcggtgacctagtcggcttgattgacacgctcgattacatcagaggcatgggcttcaagggaatctacttcgctggtacgggcttgatgaatcttccctggggctacgatggttactccccggtggataccaccctcctcgacaagcaccatggcaccctgtccgactggaggcggaccatcaaggagatccacgaccgggatatgtatgtgatcatggataatacattagcaactttgagtaacctgatcggtttcaagggccacttgaacgattcggctgatttcaatgcgaaagagtacgaggtggagtacgtcaccgagagaacgtacgccgatttccagttcagcaatgactacaatgacacctgcaactacccgaaattctggaacgagacgggatacccgttgacgtccggtggcgtgcaagacctgaagggttgctacaacagtgatttcgatcagtatggcgaactcgaggcgtttggtaatttccccgactggaagcgacagctcacgaaattcgcttcggtgcaagatcgtctgcgagaatggcacaagccggttcgcgatgtcatcacccggcattcctgcattgtgatcgccagtttggacattgatggcttccggtttgataaggccgtccaggccactctggatccgctgggtgacatgctagcggtctaccgtgaatgtgccaagcaatatggcaagaagaacttcttcctgccgggagagatcacttcgggaaacacctttggtagtctctatcttggccggggtcgtcagccaaaccagacgcccgattcggcagacgctggcgcgaagttgacgaatgcttcgtcggattcctacttcctccgcgatgatggcctgcaggcgttggacgctgcagcctttcactacaccatctatcgttcgatgacccgtttcctgggaatggacgggaacctggtggccggctttgacctgcccactgatttcatcgaagcctggaatggcatgctcgtcactaacgacttcatcaatgccttcacgggcgagttggatcccagacatatgtatggagtgtcgaaccaggataactttcgctggcccgccatcaagaatggtacggagaaatacctgctgggtctatacattgttacgctggagctgccgggaatccctttggttctctggggcgaggagcaggagatgtacgtgttcgatgcgactgcgtccaactatctgttcggtcggcaacccatgacttaccagacggcgtggtggacgcatggctgcttctcgctaaacacctcgaaattctacgatttccccaacgacaagggattgaatggttgcaacgatatcacagtgacttacgaccaacggaatccggcgcacccccttcgcaacatcatgaagcgcatgtttgagattcgggagcagtacccagtggccaatgatggcttcaaccttgagaccatttcccagcttacccaggatatctatcttccgggttcatcgactaccccgacggtgactggtctctggtcagtgctgcgcagctactaccccggggttcagaaagaggcctcgagtagcaacgatactctttggcttgtatatcataatgccaataaaacggaaacctatggcaagaactgtactagcaaggactccgctcttctgtccccttacgagtcgggcacgaagttgaaaaacctcttctatccctacgatgaactcacgctgcaggatggaccaagtgacgctagcaacggcaccgagtcctacgggtgcacgaccaacatgaaattgctgccatgggaattccgggcttacgtcaaggcgagcgacttcgttgaacctggccctaccgtcaccgagtttgtccccggccacgatgctcgcctgctgtcttccgaggatactggggaaacactgaagatccagctgggctactccaaggccatggactgcagtgccatcacgaaagcagtctccctgaattcgaccaccgtcaagggcgtcaatgccacccttgacacctctagcgtgagctgcaccaacgtcaccgccaggacgagcagtaataactacatcggcgaggttcccactgtttggacctggtccgcgaacctgacaaacgtctatcacggaatccaccagctcaccgtcaagaatgcctccacgacgtctggaacgcgcgtagatgccatcgaccgctttttattccgagtcggaacccacaccaacccgttaatatcgccactggccaactactctaccagcctcgtccatcagtcggataacggcagctactatgttcagcatgatgcggccggcgctgaccagttccgctacacgaccgactttggcctgaactggtccaactggacagactacactggtggtaacacgacgattgaatttccgacttggaccggcacagatgctcaaaagtgggagggcactcacattcgcgtccagtatttctcccgccttaccggcagcagtgattacatccaggagggtgattatgactgggaatccggtgtccctcgcagatttccccatctgtggtggaatggcccgtacaaccaatacggttatgatgccggtctggacagcaagatgcggtttgatactaaggatcgccgctggaaatatgatttcgtttatgaatggccgtccgtcggacagatcagtgtctggggcactggcaaggacggtgttccggagacaacgaatgtttatggcgatgttgacaactcatcggttgtacaggtcttgcctccctcctacttgtcgtcgaacgtgattaatatcaccgagctgccccccttcccgcaccttggctggaccatctctctcaatgatgccaacctccgctatgaattgcttccggtcggatctggatgggcccagctggtactttacatccttctctgggtcgtccctatcttgatggggctggcgggagttttcatcttcatccgcaccttctaccgtgttgagctgaataccgatggcaatgttgttaaggaggacaagctgccgctgttgttctggcgcaggatgaagggccaatctggaggtgatggccagatggaccgggaagaatcagatgtcgccgccgtggcaggcgaaatggccattgcaggagcccccgagaaacgccgcacggtcttgatcgccaccatggagtacgatattgcggattggcaggccaaggtcaagattggtggtctgggagtcatggcccagctcatgtcccaaaacctgggatatcagaatcttatctgggtggttccctgcgtgggagacatcgaatatcccgaggataccccgacagagccatacatcgtgaagatcctcgacaacccctatctcgtcaatgtgcagtatcacgttctcaacaatatcacctacgtccttcttgacgctcctgtgttccgccagcagacaaaagccgagccgtatcctccccgcatggatgacctggatagcgccatttactactccgcctggaaccaatgtatcgcagagaccatcaagcgcttcccgtccatcgatctgtatcacatcaacgatttccacggatgcctggcaccgttgtacctcctgcccacacgcacgatccccgtgtgtttgtctctccacaacgccgagttccagggtctctggcccctccggacccagcaagaaaagaaggaagtctgttcggtcttcaacctccccgtcgagacggctaccaaatattgtcaattcggtaatgtgttcaacttgcttcacacaggagcgagttaccttcggttccatcagcgcgggtttggcgcggtcggtgtgtcaaagaagtacggaaagcgttcctgggctcggtatcctattttctggagtttggacaagatcggtagtctgcccaaccctgacccaactgatacggcggccctggaggatactcctgacgagaaagctctgactcggtcatatgaggaacggatcactgacaagctcgaggcccagaagtgggctggcctgaccgaggatcgcaacgccgacctcctggtgttcgtcggtcgctggtctaagcagaagggtgtcgatctgattgcggatgtcatccccgccgttctgtctgaccggccgcaggtgcaggtgatctgtgttggtcccatcatcgatctttacggaagactggctgccattaagctggagaagatcgctgccatgttccctggccgggtcttctcccgacctgagttcactgctttgcccccgtgtgtcttctctggcgctgacttcgctctgatcccgtctcgtgatgagccatttggattgattgccgttgagttcggtcggaagggtgcactgggaatcggatctcgcatcggaggtttgggtcagatgccaggctggtggtacaccgttgaatcagacgccactcgccatcttctacaccagctgaagactgcgatcaaatctgccctggattcgactcctgaaactcgagcccagatgcgcgcaaactccgctaagcagcgattccccgttctcgagtggatccagaaactcgagactttgcagcggacatcgatcaagattcatcatgacaagaacaaggacaccgtgactggttcgatcccggagtctgagagcgtatgggacctgcaaaaccttcagagtgcgcgctattccagtgtggcacttcctaatatggctctgtccagagcggacggcacggaaaccccgcctgaagccatgatccagcaggcccacgctcgccttcaagagatccaggcatccgagggaagcagtcgcgacagcagcctcaaccgcaaactgtcactgggtcgccggtccggccctggtcagggcagaaagcgcttgacgaagaggcaagcgaccgcaagccaggcatccactgatcacggagaagatgagaacgataccgacgctgacgacgaccacaccaggccgcaggaggattacatttctcccgaggaggccatgcaggccgttaacaacactctaactccccatgctatcactggagacagtactccccgcgcctcctacttgtctagacccgtatccccgtaccccatctcacagatctctactccagcgtgcccgacaccgccacccatgactcacttccggaccgtttctatgctgtcgttgccatccgttgttggggaccataaccgcctcagcagtctcggcggcgaggagcagaaccagcccgtcttcgagttgcagaaggtggacccgactttcacggacagcttgggccacttcacccggcgcttcgagcagtcgcttgagaagttgaacaagaagaactccatcacggactattgcattgaggtctatctgatgaagagtgagcgcaagttctacaattcatacaacgacgcccagcttaagaagcagcctaagaaccgttcgctcgcggttcccagctctgagagtgttgtgcaagaagggcccggtggctcagtcgttcgtcactcgtactccagtgaggattctgaaatcaatggggaggatgagattgaccgctggctgatgcggttgggttacacgcgtccgatcgcagttcagcgattcatgagacgccgcttgggcaattggccagtgtattcactattcctgggtctgggtcagatcatcgccaccaactccgcacaaatcaccctgcttgtcggccaggtcggagagaccgctgtgaagctttatgtcatcgccgctatctactgcctgtcctccatcgcgtggtggttcctgtatcatcggttcccagccgtcatctccctcagtcttccctggttcatctactgcatggcattcatcgttatcggtgtttcacccttcggtctcacggctctcggtcgggcctgggctcagaatgttgcggctggcgtctacgccattgcgtcgtctagcggttccttgttcttcgctctgaactttggagaccagggtgccgtccctgtcaaggactggatgttccgcgccagtatcattcaaggtatctcacagatctacacggtcgcgctctggttctggagctccaaggtcactgccgccgaagtgggaggtgtatctgtagcggccttgagctcctggcgtctcacggccgtggtgatgcccattgctgccctgtgcttcgtgatcggagtcctgctggccctgggattgcccaactactaccgtcaagctcccagccgtatcctgttcttctacaagtctctgttccgccgacgcatcatcctgtggttcttcttcatggtcatcgtccagaactggttccttgctgccgctttcggccgcaactggtccttcctctggtcgtccaaacacaccaagccctgggagattgtcctccttgtgttcggcttcttcgtcgtcctatgggtagtcattcttgtcatattccgtgccctgtcgaaggaacacagctggatcttgccagttttcggtctgagtctgggtgctccccggtgggcacaaacctggtggggaacctccaacatcggctactatctcccttgggctggcagcttggtgtcggggtcgatcgcttcccgctgcctgtggctctggctgggactccttgacgagatccaacaagtcggattgggtatgattctgctgcagaccatgactcgcgtgcatgtctgcttcgtcctgttggccgcgcaagcactgggatccatcgccaccatctgcgcccgcggcttcgcgccgaacaagattggccctggggatatctctccggatgtgggcacgtcggtggacaaggtcggtaacgcatggttctggatcgctctcttcttccagctcctggcaagttggggcttcctgctcttctaccgccgcgaacagctcaaccgaccctaa

SEQ ID NO: 3 - protein, 2397 aa, aspergillus niger
MFGTTVQRCVVLILGILSVTTVGWPYDESLVDYNLNENKTAEGPINYWGEWPDHTYHPSPDNWRFPIYTIFLDRIANGDPANDDINGTTYEHVLDSNQMRHGGDLVGLIDTLDYIRGMGFKGIYFAGTGLMNLPWGYDGYSPVDTTLLDKHHGTLSDWRRTIKEIHDRDMYVIMDNTLATLSNLIGFKGHLNDSADFNAKEYEVEYVTERTYADFQFSNDYNDTCNYPKFWNETGYPLTSGGVQDLKGCYNSDFDQYGELEAFGNFPDWKRQLTKFASVQDRLREWHKPVRDVITRHSCIVIASLDIDGFRFDKAVQATLDPLGDMLAVYRECAKQYGKKNFFLPGEITSGNTFGSLYLGRGRQPNQTPDSADAGAKLTNASSDSYFLRDDGLQALDAAAFHYTIYRSMTRFLGMDGNLVAGFDLPTDFIEAWNGMLVTNDFINAFTGELDPRHMYGVSNQDNFRWPAIKNGTEKYLLGLYIVTLELPGIPLVLWGEEQEMYVFDATASNYLFGRQPMTYQTAWWTHGCFSLNTSKFYDFPNDKGLNGCNDITVTYDQRNPAHPLRNIMKRMFEIREQYPVANDGFNLETISQLTQDIYLPGSSTTPTVTGLWSVLRSYYPGVQKEASSSNDTLWLVYHNANKTETYGKNCTSKDSALLSPYESGTKLKNLFYPYDELTLQDGPSDASNGTESYGCTTNMKLLPWEFRAYVKASDFVEPGPTVTEFVPGHDARLLSSEDTGETLKIQLGYSKAMDCSAITKAVSLNSTTVKGVNATLDTSSVSCTNVTARTSSNNYIGEVPTVWTWSANLTNVYHGIHQLTVKNASTTSGTRVDAIDRFLFRVGTHTNPLISPLANYSTSLVHQSDNGSYYVQHDAAGADQFRYTTDFGLNWSNWTDYTGGNTTIEFPTWTGTDAQKWEGTHIRVQYFSRLTGSSDYIQEGDYDWESGVPRRFPHLWWNGPYNQYGYDAGLDSKMRFDTKDRRWKYDFVYEWPSVGQISVWGTGKDGVPETTNVYGDVDNSSVVQVLPPSYLSSNVINITELPPFPHLGWTISLNDANLRYELLPVGSGWAQLVLYILLWVVPILMGLAGVFIFIRTFYRVELNTDGNVVKEDKLPLLFWRRMKGQSGGDGQMDREESDVAAVAGEMAIAGAPEKRRTVLIATMEYDIADWQAKVKIGGLGVMAQLMSQNLGYQNLIWVVPCVGDIEYPEDTPTEPYIVKILDNPYLVNVQYHVLNNITYVLLDAPVFRQQTKAEPYPPRMDDLDSAIYYSAWNQCIAETIKRFPSIDLYHINDFHGCLAPLYLLPTRTIPVCLSLHNAEFQGLWPLRTQQEKKEVCSVFNLPVETATKYCQFGNVFNLLHTGASYLRFHQRGFGAVGVSKKYGKRSWARYPIFWSLDKIGSLPNPDPTDTAALEDTPDEKALTRSYEERITDKLEAQKWAGLTEDRNADLLVFVGRWSKQKGVDLIADVIPAVLSDRPQVQVICVGPIIDLYGRLAAIKLEKIAAMFPGRVFSRPEFTALPPCVFSGADFALIPSRDEPFGLIAVEFGRKGALGIGSRIGGLGQMPGWWYTVESDATRHLLHQLKTAIKSALDSTPETRAQMRANSAKQRFPVLEWIQKLETLQRTSIKIHHDKNKDTVTGSIPESESVWDLQNLQSARYSSVALPNMALSRADGTETPPEAMIQQAHARLQEIQASEGSSRDSSLNRKLSLGRRSGPGQGRKRLTKRQATASQASTDHGEDENDTDADDDHTRPQEDYISPEEAMQAVNNTLTPHAITGDSTPRASYLSRPVSPYPISQISTPACPTPPPMTHFRTVSMLSLPSVVGDHNRLSSLGGEEQNQPVFELQKVDPTFTDSLGHFTRRFEQSLEKLNKKNSITDYCIEVYLMKSERKFYNSYNDAQLKKQPKNRSLAVPSSESVVQEGPGGSVVRHSYSSEDSEINGEDEIDRWLMRLGYTRPIAVQRFMRRRLGNWPVYSLFLGLGQIIATNSAQITLLVGQVGETAVKLYVIAAIYCLSSIAWWFLYHRFPAVISLSLPWFIYCMAFIVIGVSPFGLTALGRAWAQNVAAGVYAIASSSGSLFFALNFGDQGAVPVKDWMFRASIIQGISQIYTVALWFWSSKVTAAEVGGVSVAALSSWRLTAVVMPIAALCFVIGVLLALGLPNYYRQAPSRILFFYKSLFRRRIILWFFFMVIVQNWFLAAAFGRNWSFLWSSKHTKPWEIVLLVFGFFVVLWVVILVIFRALSKEHSWILPVFGLSLGAPRWAQTWWGTSNIGYYLPWAGSLVSGSIASRCLWLWLGLLDEIQQVGLGMILLQTMTRVHVCFVLLAAQALGSIATICARGFAPNKIGPGDISPDVGTSVDKVGNAWFWIALFFQLLASWGFLLFYRREQLNRP

SEQ ID NO: 4 - DNA, 7532 bp, aspergillus niger
atgaagtgggctatttccggcacgctgctcgcctgtttcgcaacaactgcaacggcctggccttacgacgagtccctcgtcgactacaacttcaatcagaaccagtccgctacgaacccggcggactattggggaacatggcccaatcataccggggactacttcccctccccggacaattggcgcttccccgtctacaccctctttctcgaccgcttcgtcaacggtgaccccacaaacgacaacatcaatggcaccctgttcgaacatgacctccgctcgaatcagatgcgccatggtggcgatgttgccggcctgctggataccttggattacttgcagggcatgggaatcaaggtcggtccctgtggcttgtccgatgctgcgatcaattactgacgatggtttagggtctctatcttgccggaacaatcctcatgaaccagccctggggatctgacggttattcggctttggacacgacattgttggatcaacactatggtaacctgcagacgtggcgcaatgccatcacggaaattcacaagcgcgggatgtatgtcatcttcgataacaccatcgcaacgtaagtttgccgctgccttttcccctctttcctacccaacaaactaacctgcaatttttaggatgggtgatttgatcggatttgatggctatctgaacaccaccacccccttctccgtgaaggaacaccaaacggtgtggaagactgaccgccgctatgtggacttcgacattggcaatgactacaacgagacgtgcgactacccccgcttctggttcgaggatggctaccccgttcaagcgtcagtcaccgaggagcttgtcggatgttacaacagtgatttcgaccagtacggtgatattgaggcctttggtgtcttccccgattggcaacgtcagctggcaaaattcgcctccgttcaggatcgactccgtgaatgggttccctccgtgcgcgagcgtttgatccgccactcctgcatcattattcagtcgctcgatattgacggtttccggtacgacaaggcgactcaggcaaccgtcgacgccctcggagatatgtccaatgcgtaccgcgagtgcgcccgcgccgtaggaaaagagaacttcttcattgcgggcgaaatcacgggtggtaatacctttggttccatctacttgggccgaggaagacagcccaaccagttccctgactcggcggaggcagccatgaagctgaccaacacttccgacgcccagtatttcctgcgtgaagtgggacatgaggcgattgatggtgcagccttccactactccatttaccgagcgctgacccgcttcctgggcatggatggtaacttggctgccggttacgatgtgccagtcgactgggttgatgcctggaatctcatgctgcagtccaacgatctggtcaatgcgaatacaggcaagttcgacccccgccacatgtacggggctaccaaccaggatgtcttccgttggcccacggtggaaaagggcgtggagcgtcagttgcttggattgtacatcacgactcttctgctccccggaattccgctccttctctggggcgaggaacaggcgttttacgtactggatgccacggcatcgaactacatctacggtcggcaggccatgtctcctgccaccgcatggagggatcatggctgtttctccttggaatcgtctcagtactataattggcccattgagtccggccgtcagggctgccacgaccccacggtggcctacgatcatcgtgacccgtctcatcccgtgcgcaatatcattaagcatatgtatcagatgcgcgaacagttccccgtcctcaatgacggctatactattcaaaagctctcgaaccacaccgaggacgtgtactatctcggttccaacggcacagccaccgagaccggactctggtcgattcttcgtgatgtgaacgcggatgtgcaggatttgggctccgatgcgaagaaccagcctgtctggctggtttaccacaacacgaaccgcaccattgattacaagtttgactgctcggataatgatacagctctgattgcccccttcgatagtggcacctgggtcaaaaacctgttccacccgtatgacgagcaccagctgatcgattcccccaccaagttgggattgaacggatcaactgcatacagtggctgcttggctaatatgaccatgtccgcctatgaattccgggcctacgtgcctaaaacccgctttactaaacccaggccgatgattacaaagttcactccgggacacgatgtcccgatccgctcgaccgtggccccgaacgcagacgagaacgtggaggtcgaaatttacttctccgaagaaatggattgcgactcggtgacaaagtccattaccctttcgtcatcgaccgaaattggaaaggccccctctgtcgattctggcagtgtcaactgcaagtcagtccccgccactaacaccagctggaccggccagattcccggggtgtggatgtgggcggccaacctgacaggtgtgtacaacggcattcatcgtctcacagtcaacaatgtcagcacagagagtgggaacgcaaccaccaacgccgtcgaccatttcctcttccgcatcggccagattgataatccgatgattttcagcagtgcgaactattcaactagtttgctccataaggaatccaacggcacccttttcatccagcaccacgccgcgggtgctgataagtatcgctattctacgaattggggtaccactttctccgattggattgactacaggggcggaaatgacactattgaggaactcgaatggtcgggaaccaagaagcagtcatggaaaggaaaccacgttcgcgtggagtattggagtcgttggaccggcagcagcgattacgtccaagagggagacgcgggctggaacgagaatgttccacgccgtttccctcacgtcttcttcaacggaccctacaaccagtatgggtatgatgcaggtctggacaacgtggtccgccaggacagcgttgacggtctctggaaatatcatttcaccgcggaatggcctgctcaagctcaattgaacatttggggtatgaaccctgatggggagcctgaccagagttgggtcctgggagatgccgataatgattcggttctcgatcgcatgccgccctcttcgctgtccgcgacattgattaacatcactgaacaccccccctctccctacatttcgtggaatatcttcattgatgacgggaccatgcgcttccagctgttccccgtcgggcatcagaatactcagatcgccatgtatgtgctcttttggatcatccccgtcatcacgggcgcagccggtgtgtgggctttcatgaagtctttctacaaggtcaaattcaaccaggttggtgtgagcgaaaaacaccagatgatcccattggccttgcggcggaagttcaagcgcaatcgcaatcgtggcggtgatgaggaaaactcaaaccctctcatgcgtctggcgaacaagtccgggttcctccagactgacacggcgattggtggtgctgctagcggcaagcgtcgcatggtcctgatcgccaccatggagtacgacattgaggattgggccatcaagatcaagattggtggtcttggtgtcatggcgcaactcatgggtaagactctgggtcatcaagacttgatctgggtggtgccttgcgttgggggtgtcgattaccccgtggacaaacccgcagagcccatgcatgtcaccattcttggcaattcgtacgaggtccaggtccagtatcacgtcttgaataacatcacctatgttctgctggatgcccctgtgttccgtcaacagtctaagtcagagccttacccggctcgcatggacgacctgaacagcgccatttactactcggcctggaatcagtgcattgcggaagcctgcaagagattccctatcgacctgtatcatatcaacgactaccatggttccctggctccactgtatctacttcctgacacagtacctgcctgtctttccctgcataacgccgagttccaaggtttgtggcccatgcggacgcagaaagaaaaggaggaagtttgctccgtgttcaatctggatatcgagaccgtgaggcattacgtgcagtttggagaggtgttcaacttgctccactcgggtgctagttatctccgtgttcaccaacaaggtttcggtgctgttggtgtgtctaagaagtacggaaagcggtcctacgcgcgttaccccattttctggggtctccgcaaggttggaaacctacctaaccctgatccgtctgatgtcggtgagtggagcaaggaacaggccagcgccatgggtgacaatgtgagcgtggacccgacttatgaagccggtcgaggcgacctcaagcgtcaagctcaggagtgggccggtcttgaacagaaccctgacgccgatttgcttgtcttcgttggtcgttggtcgatgcagaagggtgttgatttgatcgccgacgtcatgcctgctgtcctggaagcacgccccaatgttcagctcatttgtgttggaccagttatcgatctctacggtaaattcgcggccctcaaactcgatcacatgatgaaggtctaccccggacgagtgttctctagacctgagttcacggcattgcccccctacatcttctctggtgctgaattcgcgctgattccctctcgtgacgagcccttcggtctggtcgccgtcgagttcggacgtaagggagctctgggtatcggtgcccgggttggtggtctcggtcagatgccaggttggtggtacaatgtggaatcgacagctacctcccatttgctcgttcagttcaagctggctatcgacgcggctctcagttcgaaaacggaaactcgtgctatgatgcgtgcccggtccgccaaacagcgcttcccggtcgcccagtgggtggaagacttggagatcctgcaaaccaccgccatccaagtgcacaacaaggaattggttaaacataacggtcgacccttcaccccgtctggaactaccacgccgggtggcatcttaagccagccgtcaagtccactaatgccccctggaatgcagactcccttggctcattctcgggaaagcagctattctaacctgaaccgtctcagtgaatatgtcacagatccgaaaacaaactacagtcgagacatcagccccagtgggacggaaaagccgcggtccggcctgcaacgacagctttctcttggtgttcgctcaggacctggtcatcaagagcgccgtggccgtcgtggacgccagcgcgacagcatccccgaacacgaagacaccgcaggggccatgaccgacgtcgaagaagaccacgaggacattggggatcagcaggatgcggacgacgagtacactctcaccccggctcaggtcgaggaaggacgtcgcttgcaggctgtccagcagcagggagtgggtatgccgacgagtccgggcgtccgccgttatagtcaagactccttgcatccgcgacagcttcctagcagccctggccccgtcccacctcctacacagagcctccttccgccacccaggcttggggatgccggtagccgactcagtagcgcgtctgtcctgtccctggattcggtcgttggtaccaagacggacttcaagctccagaaggttgaccccttctttacggattctactggcgagtactacaaggcattcgacaagaggctggtcggcttgaacggctcgaactccgaatcccagctttgcattgaggaatatctcatcaagagtgagaaggaatggttcgacaagttccgcgatgcccgactgggtcgtctaaaatcccccgcgtcgtctgtcttccgggacaagcatggtgcctcccctgttggctcctactacgatgatacgggctcccgggtgagtggtgactatgaccgggagtcccgcgacacggaagatgacgagttcctcctgggcaaggactacgtgcctccgacgggtctgcgcaagtggatgcagattcgtgttggagactggccagtctactcgttattccttgccttgggtcagatcattgcagccaactcttatcaggtgaccttgcttaccggtgaggttggtgagacggccgagaagctctatggtatcgcaaccacctacctgatcacctcgatcctttggtggcttgtgttccgttacttcaaatcggtggtgtgcctctcggctccctggttcttctatggccttgccttccttctgatcggctccgctcactttgaacccaattcattcaaccggggttggatccagaacatcggaagtggatgttacgccgctgcctcgtctagcggttccatcttcttcgccttgaactttggtgacgaaggtggtgcgccggtcgaaacctggatcttccgggcttgtctcattcagggtatccagtcggcttatatcatcggactgtggtattggggttccactctgaccaaggcatccagccagggtctgttgacctcgacgaacaacatcgccaatagctggaagatgacgtgagtacattgaaccctctgattgttcatcaatcagccagtcattccatcttgtgacccgctaacactgatctcctctagtgccatctgttacccgattgcaatcttcctctgggctgttggattgctgcttctctttggacttcctaactactatcgccaaaccccgggcaaagtggcctccttttacaagtccgtattccgccgcaagatcgtcctctggaacttcgttgccgtcatcctgcaaaacttcttcctcagcgcaccgtacggccgcaactggggctgtaagtctaactttccctgccacatcgtgtcgaattgataagctaactctagtccgcagtcctttggtcttccaatcacgccaaggcctggcaaatcgttattctctgtatcgtcttctacgggttcgtctgggcgggcttcctgttcgtcgtcagccgctatttcaagtcccacagctggttcctgcccgtgtttgcgtgcggacttggagcacctcgcttcattcaaatctggtggggtgtctcgggcatcggttacttccttccctgggtctccggaggctatctcggcggagctttggcctcgaggagtctctggctctggctgggcgtgttggattccatccagggtctcgggttcggtatcatcctcctgcagaccctcacccgcatgcacatgctgttcaccctgatctgctcgcaggtgcttggttccattgccaccatctgtgcgcgggcgtttgccccgaataacgtcgggcccggccctgtgtcgccggaccctacctttggaggaagtgcagtggccaatgcgtggttctgggtggccctgttttgtcagctgttggtgtgtgctggcttcctcctcttcttccggaaagagcagctttccaaaccttga

SEQ ID NO: 5 - DNA, 7278 bp, aspergillus niger
  CDS (1)..(7275): AgsE-encoding cDNA
atgaagtgggctatttccggcacgctgctcgcctgtttcgcaacaactgcaacggcctggccttacgacgagtccctcgtcgactacaacttcaatcagaaccagtccgctacgaacccggcggactattggggaacatggcccaatcataccggggactacttcccctccccggacaattggcgcttccccgtctacaccctctttctcgaccgcttcgtcaacggtgaccccacaaacgacaacatcaatggcaccctgttcgaacatgacctccgctcgaatcagatgcgccatggtggcgatgttgccggcctgctggataccttggattacttgcagggcatgggaatcaagggtctctatcttgccggaacaatcctcatgaaccagccctggggatctgacggttattcggctttggacacgacattgttggatcaacactatggtaacctgcagacgtggcgcaatgccatcacggaaattcacaagcgcgggatgtatgtcatcttcgataacaccatcgcaacgatgggtgatttgatcggatttgatggctatctgaacaccaccacccccttctccgtgaaggaacaccaaacggtgtggaagactgaccgccgctatgtggacttcgacattggcaatgactacaacgagacgtgcgactacccccgcttctggttcgaggatggctaccccgttcaagcgtcagtcaccgaggagcttgtcggatgttacaacagtgatttcgaccagtacggtgatattgaggcctttggtgtcttccccgattggcaacgtcagctggcaaaattcgcctccgttcaggatcgactccgtgaatgggttccctccgtgcgcgagcgtttgatccgccactcctgcatcattattcagtcgctcgatattgacggtttccggtacgacaaggcgactcaggcaaccgtcgacgccctcggagatatgtccaatgcgtaccgcgagtgcgcccgcgccgtaggaaaagagaacttcttcattgcgggcgaaatcacgggtggtaatacctttggttccatctacttgggccgaggaagacagcccaaccagttccctgactcggcggaggcagccatgaagctgaccaacacttccgacgcccagtatttcctgcgtgaagtgggacatgaggcgattgatggtgcagccttccactactccatttaccgagcgctgacccgcttcctgggcatggatggtaacttggctgccggttacgatgtgccagtcgactgggttgatgcctggaatctcatgctgcagtccaacgatctggtcaatgcgaatacaggcaagttcgacccccgccacatgtacggggctaccaaccaggatgtcttccgttggcccacggtggaaaagggcgtggagcgtcagttgcttggattgtacatcacgactcttctgctccccggaattccgctccttctctggggcgaggaacaggcgttttacgtactggatgccacggcatcgaactacatctacggtcggcaggccatgtctcctgccaccgcatggagggatcatggctgtttctccttggaatcgtctcagtactataattggcccattgagtccggccgtcagggctgccacgaccccacggtggcctacgatcatcgtgacccgtctcatcccgtgcgcaatatcattaagcatatgtatcagatgcgcgaacagttccccgtcctcaatgacggctatactattcaaaagctctcgaaccacaccgaggacgtgtactatctcggttccaacggcacagccaccgagaccggactctggtcgattcttcgtgatgtgaacgcggatgtgcaggatttgggctccgatgcgaagaaccagcctgtctggctggtttaccacaacacgaaccgcaccattgattacaagtttgactgctcggataatgatacagctctgattgcccccttcgatagtggcacctgggtcaaaaacctgttccacccgtatgacgagcaccagctgatcgattcccccaccaagttgggattgaacggatcaactgcatacagtggctgcttggctaatatgaccatgtccgcctatgaattccgggcctacgtgcctaaaacccgctttactaaacccaggccgatgattacaaagttcactccgggacacgatgtcccgatccgctcgaccgtggccccgaacgcagacgagaacgtggaggtcgaaatttacttctccgaagaaatggattgcgactcggtgacaaagtccattaccctttcgtcatcgaccgaaattggaaaggccccctctgtcgattctggcagtgtcaactgcaagtcagtccccgccactaacaccagctggaccggccagattcccggggtgtggatgtgggcggccaacctgacaggtgtgtacaacggcattcatcgtctcacagtcaacaatgtcagcacagagagtgggaacgcaaccaccaacgccgtcgaccatttcctcttccgcatcggccagattgataatccgatgattttcagcagtgcgaactattcaactagtttgctccataaggaatccaacggcacccttttcatccagcaccacgccgcgggtgctgataagtatcgctattctacgaattggggtaccactttctccgattggattgactacaggggcggaaatgacactattgaggaactcgaatggtcgggaaccaagaagcagtcatggaaaggaaaccacgttcgcgtggagtattggagtcgttggaccggcagcagcgattacgtccaagagggagacgcgggctggaacgagaatgttccacgccgtttccctcacgtcttcttcaacggaccctacaaccagtatgggtatgatgcaggtctggacaacgtggtccgccaggacagcgttgacggtctctggaaatatcatttcaccgcggaatggcctgctcaagctcaattgaacatttggggtatgaaccctgatggggagcctgaccagagttgggtcctgggagatgccgataatgattcggttctcgatcgcatgccgccctcttcgctgtccgcgacattgattaacatcactgaacaccccccctctccctacatttcgtggaatatcttcattgatgacgggaccatgcgcttccagctgttccccgtcgggcatcagaatactcagatcgccatgtatgtgctcttttggatcatccccgtcatcacgggcgcagccggtgtgtgggctttcatgaagtctttctacaaggtcaaattcaaccaggttggtgtgagcgaaaaacaccagatgatcccattggccttgcggcggaagttcaagcgcaatcgcaatcgtggcggtgatgaggaaaactcaaaccctctcatgcgtctggcgaacaagtccgggttcctccagactgacacggcgattggtggtgctgctagcggcaagcgtcgcatggtcctgatcgccaccatggagtacgacattgaggattgggccatcaagatcaagattggtggtcttggtgtcatggcgcaactcatgggtaagactctgggtcatcaagacttgatctgggtggtgccttgcgttgggggtgtcgattaccccgtggacaaacccgcagagcccatgcatgtcaccattcttggcaattcgtacgaggtccaggtccagtatcacgtcttgaataacatcacctatgttctgctggatgcccctgtgttccgtcaacagtctaagtcagagccttacccggctcgcatggacgacctgaacagcgccatttactactcggcctggaatcagtgcattgcggaagcctgcaagagattccctatcgacctgtatcatatcaacgactaccatggttccctggctccactgtatctacttcctgacacagtacctgcctgtctttccctgcataacgccgagttccaaggtttgtggcccatgcggacgcagaaagaaaaggaggaagtttgctccgtgttcaatctggatatcgagaccgtgaggcattacgtgcagtttggagaggtgttcaacttgctccactcgggtgctagttatctccgtgttcaccaacaaggtttcggtgctgttggtgtgtctaagaagtacggaaagcggtcctacgcgcgttaccccattttctggggtctccgcaaggttggaaacctacctaaccctgatccgtctgatgtcggtgagtggagcaaggaacaggccagcgccatgggtgacaatgtgagcgtggacccgacttatgaagccggtcgaggcgacctcaagcgtcaagctcaggagtgggccggtcttgaacagaaccctgacgccgatttgcttgtcttcgttggtcgttggtcgatgcagaagggtgttgatttgatcgccgacgtcatgcctgctgtcctggaagcacgccccaatgttcagctcatttgtgttggaccagttatcgatctctacggtaaattcgcggccctcaaactcgatcacatgatgaaggtctaccccggacgagtgttctctagacctgagttcacggcattgcccccctacatcttctctggtgctgaattcgcgctgattccctctcgtgacgagcccttcggtctggtcgccgtcgagttcggacgtaagggagctctgggtatcggtgcccgggttggtggtctcggtcagatgccaggttggtggtacaatgtggaatcgacagctacctcccatttgctcgttcagttcaagctggctatcgacgcggctctcagttcgaaaacggaaactcgtgctatgatgcgtgcccggtccgccaaacagcgcttcccggtcgcccagtgggtggaagacttggagatcctgcaaaccaccgccatccaagtgcacaacaaggaattggttaaacataacggtcgacccttcaccccgtctggaactaccacgccgggtggcatcttaagccagccgtcaagtccactaatgccccctggaatgcagactcccttggctcattctcgggaaagcagctattctaacctgaaccgtctcagtgaatatgtcacagatccgaaaacaaactacagtcgagacatcagccccagtgggacggaaaagccgcggtccggcctgcaacgacagctttctcttggtgttcgctcaggacctggtcatcaagagcgccgtggccgtcgtggacgccagcgcgacagcatccccgaacacgaagacaccgcaggggccatgaccgacgtcgaagaagaccacgaggacattggggatcagcaggatgcggacgacgagtacactctcaccccggctcaggtcgaggaaggacgtcgcttgcaggctgtccagcagcagggagtgggtatgccgacgagtccgggcgtccgccgttatagtcaagactccttgcatccgcgacagcttcctagcagccctggccccgtcccacctcctacacagagcctccttccgccacccaggcttggggatgccggtagccgactcagtagcgcgtctgtcctgtccctggattcggtcgttggtaccaagacggacttcaagctccagaaggttgaccccttctttacggattctactggcgagtactacaaggcattcgacaagaggctggtcggcttgaacggctcgaactccgaatcccagctttgcattgaggaatatctcatcaagagtgagaaggaatggttcgacaagttccgcgatgcccgactgggtcgtctaaaatcccccgcgtcgtctgtcttccgggacaagcatggtgcctcccctgttggctcctactacgatgatacgggctcccgggtgagtggtgactatgaccgggagtcccgcgacacggaagatgacgagttcctcctgggcaaggactacgtgcctccgacgggtctgcgcaagtggatgcagattcgtgttggagactggccagtctactcgttattccttgccttgggtcagatcattgcagccaactcttatcaggtgaccttgcttaccggtgaggttggtgagacggccgagaagctctatggtatcgcaaccacctacctgatcacctcgatcctttggtggcttgtgttccgttacttcaaatcggtggtgtgcctctcggctccctggttcttctatggccttgccttccttctgatcggctccgctcactttgaacccaattcattcaaccggggttggatccagaacatcggaagtggatgttacgccgctgcctcgtctagcggttccatcttcttcgccttgaactttggtgacgaaggtggtgcgccggtcgaaacctggatcttccgggcttgtctcattcagggtatccagtcggcttatatcatcggactgtggtattggggttccactctgaccaaggcatccagccagggtctgttgacctcgacgaacaacatcgccaatagctggaagatgactgccatctgttacccgattgcaatcttcctctgggctgttggattgctgcttctctttggacttcctaactactatcgccaaaccccgggcaaagtggcctccttttacaagtccgtattccgccgcaagatcgtcctctggaacttcgttgccgtcatcctgcaaaacttcttcctcagcgcaccgtacggccgcaactggggcttcctttggtcttccaatcacgccaaggcctggcaaatcgttattctctgtatcgtcttctacgggttcgtctgggcgggcttcctgttcgtcgtcagccgctatttcaagtcccacagctggttcctgcccgtgtttgcgtgcggacttggagcacctcgcttcattcaaatctggtggggtgtctcgggcatcggttacttccttccctgggtctccggaggctatctcggcggagctttggcctcgaggagtctctggctctggctgggcgtgttggattccatccagggtctcgggttcggtatcatcctcctgcagaccctcacccgcatgcacatgctgttcaccctgatctgctcgcaggtgcttggttccattgccaccatctgtgcgcgggcgtttgccccgaataacgtcgggcccggccctgtgtcgccggaccctacctttggaggaagtgcagtggccaatgcgtggttctgggtggccctgttttgtcagctgttggtgtgtgctggcttcctcctcttcttccggaaagagcagctttccaaaccttga

SEQ ID NO: 6 - protein, 2425 aa, aspergillus niger
MKWAISGTLLACFATTATAWPYDESLVDYNFNQNQSATNPADYWGTWPNHTGDYFPSPDNWRFPVYTLFLDRFVNGDPTNDNINGTLFEHDLRSNQMRHGGDVAGLLDTLDYLQGMGIKGLYLAGTILMNQPWGSDGYSALDTTLLDQHYGNLQTWRNAITEIHKRGMYVIFDNTIATMGDLIGFDGYLNTTTPFSVKEHQTVWKTDRRYVDFDIGNDYNETCDYPRFWFEDGYPVQASVTEELVGCYNSDFDQYGDIEAFGVFPDWQRQLAKFASVQDRLREWVPSVRERLIRHSCIIIQSLDIDGFRYDKATQATVDALGDMSNAYRECARAVGKENFFIAGEITGGNTFGSIYLGRGRQPNQFPDSAEAAMKLTNTSDAQYFLREVGHEAIDGAAFHYSIYRALTRFLGMDGNLAAGYDVPVDWVDAWNLMLQSNDLVNANTGKFDPRHMYGATNQDVFRWPTVEKGVERQLLGLYITTLLLPGIPLLLWGEEQAFYVLDATASNYIYGRQAMSPATAWRDHGCFSLESSQYYNWPIESGRQGCHDPTVAYDHRDPSHPVRNIIKHMYQMREQFPVLNDGYTIQKLSNHTEDVYYLGSNGTATETGLWSILRDVNADVQDLGSDAKNQPVWLVYHNTNRTIDYKFDCSDNDTALIAPFDSGTWVKNLFHPYDEHQLIDSPTKLGLNGSTAYSGCLANMTMSAYEFRAYVPKTRFTKPRPMITKFTPGHDVPIRSTVAPNADENVEVEIYFSEEMDCDSVTKSITLSSSTEIGKAPSVDSGSVNCKSVPATNTSWTGQIPGVWMWAANLTGVYNGIHRLTVNNVSTESGNATTNAVDHFLFRIGQIDNPMIFSSANYSTSLLHKESNGTLFIQHHAAGADKYRYSTNWGTTFSDWIDYRGGNDTIEELEWSGTKKQSWKGNHVRVEYWSRWTGSSDYVQEGDAGWNENVPRRFPHVFFNGPYNQYGYDAGLDNVVRQDSVDGLWKYHFTAEWPAQAQLNIWGMNPDGEPDQSWVLGDADNDSVLDRMPPSSLSATLINITEHPPSPYISWNIFIDDGTMRFQLFPVGHQNTQIAMYVLFWIIPVITGAAGVWAFMKSFYKVKFNQVGVSEKHQMIPLALRRKFKRNRNRGGDEENSNPLMRLANKSGFLQTDTAIGGAASGKRRMVLIATMEYDIEDWAIKIKIGGLGVMAQLMGKTLGHQDLIWVVPCVGGVDYPVDKPAEPMHVTILGNSYEVQVQYHVLNNITYVLLDAPVFRQQSKSEPYPARMDDLNSAIYYSAWNQCIAEACKRFPIDLYHINDYHGSLAPLYLLPDTVPACLSLHNAEFQGLWPMRTQKEKEEVCSVFNLDIETVRHYVQFGEVFNLLHSGASYLRVHQQGFGAVGVSKKYGKRSYARYPIFWGLRKVGNLPNPDPSDVGEWSKEQASAMGDNVSVDPTYEAGRGDLKRQAQEWAGLEQNPDADLLVFVGRWSMQKGVDLIADVMPAVLEARPNVQLICVGPVIDLYGKFAALKLDHMMKVYPGRVFSRPEFTALPPYIFSGAEFALIPSRDEPFGLVAVEFGRKGALGIGARVGGLGQMPGWWYNVESTATSHLLVQFKLAIDAALSSKTETRAMMRARSAKQRFPVAQWVEDLEILQTTAIQVHNKELVKHNGRPFTPSGTTTPGGILSQPSSPLMPPGMQTPLAHSRESSYSNLNRLSEYVTDPKTNYSRDISPSGTEKPRSGLQRQLSLGVRSGPGHQERRGRRGRQRDSIPEHEDTAGAMTDVEEDHEDIGDQQDADDEYTLTPAQVEEGRRLQAVQQQGVGMPTSPGVRRYSQDSLHPRQLPSSPGPVPPPTQSLLPPPRLGDAGSRLSSASVLSLDSVVGTKTDFKLQKVDPFFTDSTGEYYKAFDKRLVGLNGSNSESQLCIEEYLIKSEKEWFDKFRDARLGRLKSPASSVFRDKHGASPVGSYYDDTGSRVSGDYDRESRDTEDDEFLLGKDYVPPTGLRKWMQIRVGDWPVYSLFLALGQIIAANSYQVTLLTGEVGETAEKLYGIATTYLITSILWWLVFRYFKSVVCLSAPWFFYGLAFLLIGSAHFEPNSFNRGWIQNIGSGCYAAASSSGSIFFALNFGDEGGAPVETWIFRACLIQGIQSAYIIGLWYWGSTLTKASSQGLLTSTNNIANSWKMTAICYPIAIFLWAVGLLLLFGLPNYYRQTPGKVASFYKSVFRRKIVLWNFVAVILQNFFLSAPYGRNWGFLWSSNHAKAWQIVILCIVFYGFVWAGFLFVVSRYFKSHSWFLPVFACGLGAPRFIQIWWGVSGIGYFLPWVSGGYLGGALASRSLWLWLGVLDSIQGLGFGIILLQTLTRMHMLFTLICSQVLGSIATICARAFAPNNVGPGPVSPDPTFGGSAVANAWFWVALFCQLLVCAGFLLFFRKEQLSKP

SEQ ID NO: 7 - DNA, 30 bp, artificial sequence
  Primer agsA1 (sense)
ggtggcggccgcctgtgaatagctaccagc

SEQ ID NO: 8 - DNA, 30 bp, artificial sequence
  Primer agsA2 (antisense)
ctccactagttgctaatgtattatccatga

SEQ ID NO: 9 - DNA, 30 bp, artificial sequence
  Primer agsA3 (sense)
cttctagagtttaaacaaccgcctcagcag

SEQ ID NO: 10 - DNA, 30 bp, artificial sequence
  Primer agsA4 (antisense)
ttaattaaagaagggcattctatggacgta

SEQ ID NO: 11 - DNA, 28 bp, artificial sequence
  Primer agsE1 (sense)
accgcggtctttcagccagggaatggct

SEQ ID NO: 12 - DNA, 30 bp, artificial sequence
  Primer agsE2 (antisense)
ttactagtacacaatagtgcaggtgatgtc

SEQ ID NO: 13 - DNA, 29 bp, artificial sequence
  Primer agsE3 (sense)
tttctagacgccttaatgatgtggtggtg

SEQ ID NO: 14 - DNA, 28 bp, artificial sequence
  Primer agsE4 (antisense)
ttaattaaatggcgcccactgctacagg

SEQ ID NO: 15 - DNA, 20 bp, artificial sequence
  Forward primer (agsA5)
agcagtctcggcggcgagga

SEQ ID NO: 16 - DNA, 20 bp, artificial sequence
  Reverse primer (agsA6)
attgcccaagcggcgtctca

SEQ ID NO: 17 - DNA, 20 bp, artificial sequence
  Forward primer (agsE5)
accgggcatgaacatttaac

SEQ ID NO: 18 - DNA, 20 bp, artificial sequence
  Reverse primer (agsE6)
aagtatttattgtattttgg

SEQ ID NO: 19 - DNA, 33 bp, artificial sequence
  Primer ap025-1 (sense)
tttggatccaccatgcgtttcatttctgtctcc

SEQ ID NO: 20 - DNA, 30 bp, artificial sequence
  Primer ap025-2 (antisense)
ccacgtgttagcaaccaaggtatatggcat

SEQ ID NO: 21 - DNA, 20 bp, artificial sequence
  Forward primer (ap025-3)
aggatcagttcctgctccgg

SEQ ID NO: 22 - DNA, 20 bp, artificial sequence
  Reverse primer (ap025-4)
ggtatatggcattcgcatag

SEQ ID NO: 23 - DNA, 20 bp, artificial sequence
  Forward primer (ap025-3)
aggatcagttcctgctccgg

SEQ ID NO: 24 - DNA, 20 bp, artificial sequence
  Reverse primer (ap025-4)
ggtatatggcattcgcatag